(19) United States
(12) Patent Application Publication
(10) Pub. No.: US 2005/0222681 A1
(43) Pub. Date: Oct. 6, 2005

(54) Devices and methods for minimally invasive treatment of degenerated spinal discs

(76) Inventors: Richard Richley, San Diego, CA (US); Marvin P. Loeb, Huntington Beach, CA (US)

Correspondence Address: OLSON & HIERL, LTD., 20 NORTH WACKER DRIVE, 36TH FLOOR, CHICAGO, IL, 60606, US

(21) Appl. No.: 10/518,423

(22) PCT Filed: Jun. 17, 2003
(86) PCT No.: PCT/US03/19150

§ 371 Date: Dec. 17, 2004

Related U.S. Application Data
(60) Provisional application No. 60/389,365, filed on Jun. 17, 2002.

(51) Int. Cl. A61F 2/44

(52) U.S. Cl. 623/17.11; 623/908

(57) ABSTRACT

Spinal stabilization devices and their methods of insertion and use to treat degenerated lumbar, thoracic or cervical spinal discs in minimally invasive, outpatient procedures are described. In one embodiment, the spinal stabilization device is an expandable cage made of a coil or perforated cylindrical tube with a bulbous or bullet-shaped distal end and a flat or rounded proximal end. In a preferred embodiment, the spinal stabilization device is mechanically expanded to a larger diameter or is made of a superelastic nickel-titanium alloy which is thermally programmed to expand to a relatively larger diameter when a pre-determined transition temperature below body temperature is reached. To treat a degenerated disc, a guide wire is inserted into the disc and an endoscope is inserted through a posterolateral puncture in the back and advanced up to the facet of the spine. Mechanical tools or laser energy, under endoscopic visualization, are used to remove or vaporize a portion of the facet bone, creating an opening into the foraminal space in the spine for insertion of an endoscope, which enables the disc, vertebra and nerves to be seen. The passageway is expanded, mechanical tools or laser of RF energy are used to make a tunnel into the disc, and a delivery cannula is inserted up to the opening of the tunnel. An insertion tool is used to insert one or more spinal stabilization devices into the tunnel in the disc, preserving the mobility of the spine, while maintaining the proper space between the vertebra. Laser or radio frequency (RF) energy is used to coagulate bleeding, vaporize or remove debris and shrink the annulus of the disc to close, at least partially, the tunnel made in the disc.

### FIELD OF THE INVENTION

The present invention relates to devices and methods for the treatment of degenerated spinal discs.

### BACKGROUND OF THE INVENTION

Spinal discs which have degenerated due to disease, injury, deformity or old age (dehydration) cause severe, debilitating back, leg and neck pain. The surgical treatment of degenerated spinal discs in the United States costs about $24 billion each year. Doctors' office visits, pain killers, steroids, traction and, most importantly, absences from work add many more billions of cost annually.

Lower back pain, which often radiates into the legs, affects an estimated 15 million people in the United States and is the principal reason for absences from work. Lower back pain arises from several conditions, the most common causes being a herniated disc, in which the annulus fibrosis or fibrous exterior of the disc has bulged outward and is pressing against the nerves in the spine, a ruptured disc, whose jelly-like nucleus pulposa has been extruded through a rupture in the annulus of the disc and is pressing against the nerves in the spine, or a degenerated disc, which no longer provides a resilient cushion between the vertebra and allows the vertebra to press upon or pinch one or more of the nerves which lie along the posterior of the disc. Some persons with degenerated discs due to arthritis also often suffer from degenerated articular processes, the bony extensions of the vertebra which function as anchors and joints of the spine, and which may have deteriorated due to injuries, inflammation, arthritis or advanced age.

The spine is divided into three sections, lumbar or lower spine, terminating in the sacrum, thoracic or upper-spine and cervical or neck. Discs in these sections are likewise called lumbar, thoracic or cervical discs. A diseased, damaged or degenerated spinal disc is first treated conservatively, which entails bed rest, pain killers, injections of cortisone or other non-steroidal anti-inflammatory drugs, traction and the like. If the pain is not relieved and becomes unbearable, conventional disc surgery is the usual treatment.

While a herniated or bulging lumbar disc may be treated in a conventional open surgical procedure, a herniated lumbar disc may also be treated in a minimally invasive, outpatient laser procedure. In the latter, after administration of a local anesthetic, a side-firing laser needle, such as the Spinal MAX™ side-firing laser needle manufactured by Trimedyne, Inc. (Irvine, Calif.), may be inserted posterolaterally into the back through a small puncture and guided into the herniated lumbar disc under x-ray imaging. The emission port of the laser needle may be aimed toward the herniation, and laser energy, such as generated at a wavelength of 2100 microns by the OmniPulse™ MAX 80 watt Holmium laser manufactured by Trimedyne, Inc. (Irvine, Calif.), may be transmitted through the laser needle to vaporize and shrink a portion of the nucleus pulposa of the disc to relieve the pressure on the disc's distended or bulged annulus, the tough exterior of the disc. The side-firing laser needle and its method of use are described in co-owned U.S. Pat. No. 5,649,924 to Everett, et al, and No. 5,437,660 to Johnson et al, respectively, which are fully incorporated herein by reference.

However, when a portion of the nucleus pulposa has been expelled through a rupture in the annulus of a lumbar disc and is pressing against the nerves in the spine, an intra-discal therapy cannot be used. While a conventional surgical procedure could be performed, a minimally invasive, outpatient, laser procedure may be employed, using an endoscope, such as the OmniView™ endoscope marketed by Trimedyne Inc. (Irvine, Calif.), or the KESS® or YESS® endoscopes manufactured by Richard Wolf Instruments, Ltd. (Knittlingen, Germany). The endoscope may be inserted, posterolaterally into the back through a small puncture, as described above. The Spinal MAX™ side firing laser needle may be passed through a channel of the endoscope, and laser energy from the Omnipulse™ MAX Holmium laser may be transmitted through the laser needle to vaporize a non-load bearing portion of the bone of the facet (a bony projection of the vertebra). This creates an opening for the endoscope into the foraminal space in the spine, enabling the vertebra, disc, nerves and extruded pulposa to be seen. Laser energy, RF energy or mechanical tools may then be used to vaporize or remove any intervening tissue and vaporize or remove the extruded disc pulposa. This procedure is referred to as an endoscopic laser foraminoplasty or “ELF” procedure. The laser needle or an RF energy emitting device may also be inserted into the disc and, at a lower energy level, used to shrink a portion of the intact nucleus pulposa of the disc to create a more dense body and reduce the pressure on the annulus, as well as to shrink the annulus of the disc to reduce or close the rupture.

To treat a degenerated lumbar disc, surgeons usually employ a posterior approach, performing an open surgical “fusion” procedure. This procedure, which entails general anesthesia, a hospital stay of several days, significant post-operative pain and a recovery period of several months, is performed through a sizeable incision in the back. After the back has been opened, the intervening articular processes (bony extensions of the vertebra) are surgically removed with a mechanical tool, such as a chisel, auger, rongeur or rotating burr or shaver to gain access to the disc. A spreading device is inserted between the vertebra to hold them apart, and the diseased disc is completely or partially removed with mechanical tools, such as graspers or a rotating shaver, burr or auger. Generally, autologous or cadaver bone plugs or spacers or one or two hollow, perforated cylindrical or ovoid metal tubes or coils, typically made of titanium or a nickel titanium alloy, called “cages”, such as the InterFix® cage manufactured by Medtronic Sofamor Danek, Inc. (Nashville, Tenn.), are inserted into the space between the vertebra, and the spreading device is removed.

If the surgeon wishes to achieve fusion of the vertebra above and below the degenerated disc, he may select a cage with an outside diameter larger than the intervertebral space. A portion of the end plates and cancellous bone of the vertebra is removed before or as a part of the process of inserting the cage. The cage may be packed with autologous or cadaver bone chips or plugs and, optionally, with bone-growth stimulating materials to promote bone ingrowth from the vertebra into the bone chips or plugs packed in the cage, to immobilize that portion of the spine. However, removing bone from the vertebra to accommodate the larger cage causes bleeding and can weaken the vertebra and result in fractures, with significant adverse results.

Rods and screws, often made of titanium or a titanium-nickel alloy, are attached to the pedicles of the vertebra, above and below the diseased disc, to maintain the space between the vertebra. Immobilizing a portion of the spine often causes damage to occur over time to the discs above and below the immobilized vertebra, which frequently requires one or more subsequent surgical procedures. Approximately 400,000 of such spinal fusion surgeries are performed each year in the United States at a cost of about $60,000 each, resulting in an aggregate cost to the U.S. healthcare system of about $24 billion per year.

Recently, anterior and anterolateral surgical procedures have been developed to treat degenerated lumbar discs, which avoid removing the articular processes of the vertebra, that prevent access to the disc from a posterior approach. In these anterior or anterolateral surgical procedures, a sizeable incision is made in the abdomen and the intervening organs, the aorta and other blood vessels are moved (retracted) away from the area of the disc to be treated, the vertebra are spread apart and autologous or cadaver bone plugs or spacers or one or more cages are inserted, which may be packed with bone, as described above.

Anterior and anterolateral laparoscopic procedures also have been developed, to treat a degenerated lumbar disc, in which the procedure is performed through two or more punctures in the abdomen. An endoscope is inserted through one puncture and various tools are inserted through the others. The abdomen is expanded with a gas, such as carbon dioxide, the intervening organs, the aorta and other blood vessels are moved (retracted) away from the area of the disc, the vertebra are spread apart and one or more bone plugs or cages, which may be packed with bone, as described above, are inserted in a procedure similar to the aforementioned anterior surgical procedure.

In addition to open “surgical fusion” procedures, using a posterior approach, as described above, lower degenerated thoracic discs can also be treated in a thoracoscopic, arterolateral procedure with one of the lungs deflated and other organs and blood vessels retracted. Cages, which may be packed with bone or bone plugs, may be inserted, as described above, in a manner similar to the aforementioned anterior and anterolateral surgical procedures. However, degenerated, upper thoracic discs, at the level of the heart, cannot be so treated.

To treat a degenerated cervical disc, an incision is usually made in the neck, anterolaterally, and the larynx, esophagus, carotid artery, jugular vein and other tissues are moved away. Again, the vertebra are spread apart, all or part of the disc is removed, bone plugs or cages, often packed with bone, are inserted, as described above.

While herniated or ruptured lumbar, thoracic and cervical discs can presently be treated in minimally invasive, outpatient procedures, degenerated lumbar, thoracic or cervical discs cannot be so treated. The disadvantages of the current surgical procedures to treat degenerated lumbar, thoracic and cervical discs are the need for a large incision or several smaller incisions, the risk of infections, the risk of damage to intervening organs, arteries, veins, nerves and other tissues, the risk of general anesthesia, the cost and inconvenience of hospitalization, substantial bleeding, significant post-operative pain, a lengthy recuperation period, often 2-3 months or longer, and a substantial failure rate, as well as the need for subsequent surgery to treat the discs above and below the immobilized vertebra.

Consequently, it would be desirable to be able to treat a degenerated lumbar, thoracic or cervical disc in a minimally invasive, outpatient procedure, reducing the risks, morbidity and cost of traditional surgical procedures and reducing the failure rate. It would also be desirable to do so without immobilizing the spine, providing more normal spinal movement for the patient and reducing the need for subsequent surgeries.

### SUMMARY OF THE INVENTION

The spinal stabilization device of the present invention includes a cage made of a coil of wire or a perforated cylinder, which may have anchors such as ridges or threads on its exterior, with a bullet shaped distal end and an end plate covering its proximal end. The cage is usually made of titanium or a nickel-titanium alloy. The cage may also be made of a strong, resilient material, such as a carbon fiber, reinforced plastic or high density polyethylene. For use in a degenerated lumbar disc, the spinal stabilization device can have an outside diameter of about 6 to 14 mm, preferably about 8 to 12 mm, and can be about 20 to 30 mm in length, preferably about 23 to 27 mm long.

In a preferred embodiment, the spinal stabilization device of the present invention comprises an expandable cage having a closed, rounded distal end and an open proximal end. The cage is provided with plural anchors on the external surface thereof for engagement with contiguous bone tissue when the cage is expanded. The cage may be made of a memory metal which expands to a predetermined shape at body temperature, or the cage may be expandable mechanically.

A preferred method for stabilizing the spine of a human patient comprises the steps of forming a passageway in a spinal disc, inserting an expandable spinal stabilization device into the formed passageway, and expanding the inserted spinal stabilization device while in the formed passageway sufficient to stabilize the spine.

While this invention is susceptible of embodiment in many different forms, there are shown in the drawings and will be described in detail herein specific embodiments thereof, with the understanding that the present disclosure is to be considered as an exemplification of the principles of the invention and is not to be limited to the specific embodiments illustrated.

In the present invention, to treat a degenerated lumbar spinal disc or a degenerated lower thoracic disc in a minimally invasive, outpatient procedure, a posterolateral approach may be used. To treat an degenerated upper thoracic disc, unless the heart and major blood vessels obstruct access to the disc, or a degenerated cervical disc in a minimally invasive, outpatient procedure, an arterolateral approach may be utilized. In the procedures contemplated by the present invention, there is no need to destroy the bony, articular processes of the spine, immobilize the vertebra above and below the degenerated disc with rods and screws, remove a portion of the end plates and cancellous bone of the vertebra, or move intervening organs and blood vessels away from the area to be treated.

While disc sizes vary widely, for example, using the skeleton of an averaged sized, 60 year old male, the height of the disc space, between lumbar vertebra, was about 8 to 10 mm, and the disc, seen laterally from the side, was about 40 to 45 mm deep and about 45 to 60 mm wide.

Likewise, the height of the disc space, between thoracic vertebra, was about 5 to 8 mm, with a depth usually of about 30 to 40 mm for lower thoracic discs, a depth of about 15 to 30 mm for upper thoracic discs, and a width somewhat wider than their depth.

Similarly, the height of the disc space, between cervical vertebra, was about 3 to 5 mm, with a depth usually of about 12 to 18 mm for lower cervical discs, a depth of about 7 to 12 mm for upper cervical discs, and a width somewhat larger than their depth.

While a spinal stabilization device larger than the disc space can be inserted, which requires removal of a portion of the end plates and cancellous bone of the vertebra above and below the degenerated disc, subjecting the patient to the risk of a fractured vertebra, it is preferred to utilize a spinal stabilization device equal to or slightly smaller in diameter than the normal disc space, which the surgeon may determine based on x-rays and observation of the patient's anatomy.

To treat a degenerated lumbar disc, after injection of a local anesthetic, a puncture is made posterolaterally, approximately 10 to 15 cm from the midline of the back, preferably about 11 to 14 cm, at the level of the disc to be treated, with a scalpel, trocar, stylus, or other tool, as known in the art. A guidewire is inserted, additional local anesthetic is instilled and, under x-ray or fluoroscopic imaging, the guidewire, is advanced into the disc. Alternatively, a hollow needle may be inserted into the disc, a guidewire may be inserted through the needle, and the needle may be removed.

Dilating cannulas of increasing diameter are introduced over the guidewire until a passageway of about 3 to 6 mm in diameter, preferably about 4 to 5 mm, has been created up to the facet. An endoscope is inserted over the guidewire into the passageway and likewise advanced up to the facet. The guidewire is removed and a mechanical tool, such as a rasp, rotating shaver, burr or auger, or a laser energy transmitting device is inserted through the endoscope and used to remove a small amount of facet bone (usually the inferior facet bone, but sometimes the superior facet bone) to create a passageway for the endoscope into the foraminal space in the spine. However, mechanical tools cause significant debris in removing bone, requiring extensive flushing to remove them, and their complete removal cannot be assured.

Preferably a side firing laser needle, such as described above, whose proximal end is optically coupled to an appropriate source of pulsed laser energy, preferably a Holmium laser such as described above, is employed to vaporize a portion of the facet bone, as its use does not thermally damage and weaken the remaining bone, and bone debris is eliminated. The endoscope is advanced through the opening into the foraminal space, enabling the disc, vertebra and the traversing, exiting and other nerves to be seen. Electrocautery, RF energy or laser energy may be used to coagulate any bleeding. Laser energy is preferred; as this avoids having to switch or exchange the laser needle with an RF or electrocautery device.

The guidewire is again inserted through the endoscope, and the endoscope is withdrawn, leaving the guidewire in place. Dilating cannulas of increasing size are inserted over the guidewire, expanding the passageway to a diameter of about 6 to 14 mm, preferably about 8 to 12 mm, to accommodate a spinal stabilization device whose outside diameter is equal to or slightly smaller in diameter than the disc space. A delivery cannula, whose inside diameter is slightly larger than the spinal stabilization device which the surgeon has selected to be later inserted into the disc, is inserted into the expanded passageway and advanced up to the disc. A dye, which preferably stains degenerated disc tissue blue or another contrasting color, may be injected into the disc to aid in the visual identification of degenerated disc tissue.

Mechanical tools, such as a rotating reamer, a disc space cutter or a disc space debrider, as known in the art, are used to remove a portion of the annulus fibrosus and nucleus pulposa of the disc, creating a tunnel of about 6 to 14 mm in diameter, preferably about 8 to 12 mm, into the disc space. Additional degenerated disc material, identified by the dye, may also be removed. The end plates above and below the disc space may be injected with xylocalne or epinephrine and lightly scraped to stimulate the transfer of oxygen and nutrients to the disc.

For use in a degenerated thoracic disc, the tunnel into the disc and the spinal stabilization device can have an outside diameter of about 4 to 10 mm, preferably about 5 to 8 mm in diameter. For use in a degenerated lower thoracic disc, the spinal stabilization device can be about 17 to 25 mm in length, preferably about 19 to 23 mm long. For use in a degenerated upper thoracic disc, the device can be about 10 to 20 mm in length, preferably about 12 to 17 mm long.

For use in a degenerated cervical disc, the spinal stabilization device can have an outside diameter of about 2 to 7 mm, preferably about 3 to 6 mm. For use in a degenerated lower cervical disc, the device can be about 8 to 14 mm in length, preferably about 10 to 12 mm long. For use in a degenerated upper cervical disc, the device can be about 5 to 10 mm in length, preferably about 6 to 9 mm long.

If the patient's anatomy requires, larger or smaller diameter or longer or shorter spinal stabilization devices may be utilized. If the outside diameter of the spinal stabilization device selected by the surgeon, with an intent to create fusion by bone ingrowth from the vertebra into bone packed in the cage of the spinal stabilization device, will require the removal of a portion of the end plates and cancellous bone of the vertebra, the cage of the spinal stabilization device may have an outside diameter up to 50% larger than the sizes described above, but with the same lengths as shown above.

The spinal stabilization device is removably attached to the distal end of a screwing or insertion device, which may consist of a handle and a shaft with a key or pin at its distal end, which can be removably be inserted into shaft and key slots of the end plate of the spinal stabilization device, as described below. However, other configurations of the end plate and screwing device can be employed, such as a hexagonal recess in the proximal end plate of the cage and a hexagonal ended screwing device, as known in the art.

The spinal stabilization device, removably attached to the screwing or insertion device, is inserted through the delivery cannula and, under x-ray or fluroscopic imaging, is tapped or screwed into a place in the tunnel made earlier in the disc, using about 70 to 110 ft.-lbs. of torque. Preferably, the spinal stabilization device is centered in the disc. When the spinal stabilization device has been properly positioned, the key on the shaft of the screwing device is aligned with the key slot in the proximal end plate of the spinal stabilization device, and the screwing device is removed.

The endoscope may then be reinserted through the delivery cannula and mechanical tools or laser or RF energy may be used to remove any debris. RF or laser energy may also be used to coagulate any bleeding and to shrink the annulus of the disc to close, at least partially, the opening made in the annulus, and the delivery cannula is removed.

Usually, only a single stitch and an adhesive bandage, such as a Band-Aid® made by Johnson & Johnson (New Brunswick, N.J.), is applied to the puncture, the patient walks out of the hospital or surgery center and is able to return to light activities in a few days (light manual labor in about 2 to 10 weeks). General anesthesia is not required, the risk of infection is lessened and post operative pain is significantly reduced. Since a hospital stay and subsequent physical therapy/rehabilitation are eliminated, the cost of the procedure is reduced to less than one-half of the cost of the aforementioned posterior surgical fusion procedure or anterior or arterolateral surgical or laparoscopic procedures.

In a preferred embodiment, for use in a degenerated lumbar disc, a mechanically expandable, bird cage-type spinal stabilization device with an outside diameter of about 4 to 10 mm, preferably about 5 to 8 mm, prior to its expansion, is inserted through a delivery cannula with an inside diameter slightly larger than the outside diameter of the unexpanded, cage-type spinal stabilization device. This reduces the size of the tunnel made into the disc space to about 4 mm to 10 mm, preferably to about 5 to 8 mm. However, if the surgeon wishes to utilize an expandable spinal stabilization device larger than the disc space, the diameter of the tunnel may be commensurately larger.

The mechanically expandable spinal stabilization device, which is preferably made of a nickel-titanium alloy, such as nitinol, may have a smooth or rough textured exterior or, optionally, may have continuous or interrupted threads about its exterior, and can be removably attached to the aforementioned screwing device, as described above. The expandable spinal stabilization device, prior to its expansion, is screwed or tapped into place in the disc, as described above, after which it is mechanically expanded, by rotating the screwing device, until the device has been mechanically expanded to a desired outside diameter (usually ascertained by x-ray imaging).

For use in a degenerated lumbar disc, when expanded, the mechanically expandable spinal stabilization device may have an outside diameter of about 6 to 14 mm, preferably about 8 to 12 mm and can be about 20 to 30 mm in length, preferably about 23 to 27 mm long.

For use in a degenerated thoracic disc, the mechanically expandable spinal stabilization device can be inserted through a posterolateral or anterolateral approach and can have an outside diameter, prior to its expansion, of about 2 to 7 mm, preferably about 3 to 6 mm. When expanded, the expandable spinal stabilization device for use in a degenerated thoracic disc may have an expanded outside diameter of about 4 to 10 mm, preferably about 5 to 8 mm. The spinal stabilization device, for use in a degenerated lower thoracic disc, can be about 17 to 25 mm in length, preferably about 19 to 23 mm long, and for use in a degenerated upper thoracic disc, the device can be about 10 to 20 mm long, preferably about 12 to 17 mm in length.

For use in a degenerated cervical disc, inserted from an anterolateral approach, as described above, the tunnel into the disc and the expandable spinal stabilization device, prior to its expansion, can have an outside diameter of about 2 to 5 mm in diameter, preferably about 3 to 4 mm. When expanded, the device may have an outside diameter of 4 to 7 mm, preferably about 3 to 6 mm. For use in a degenerated lower cervical disc, the length of the device can be about 8 to 14 mm, preferably about 10 to 13 mm long. For use in a degenerated upper cervical disc, the length of the device can be about 5 to 10 mm long, preferably about 6 to 9 mm in length.

Alternatively, the bird cage-type spinal stabilization device can be made of a superelastic, shaped-memory, nickel-titanium alloy called memory metal, which is manufactured by several companies, including Memry, Inc. (Bethel, Conn.). The expandable spinal stabilization device made of memory metal may have been earlier heat treated to expand to its desired expanded outside diameter, when its temperature reaches a selected transition temperature, for example, about 65° to 90° F. The pre- and post-expansion diameters of the superelastic memory metal devices and their length can be the same as those described above for the mechanically expanded devices.

The ratio of the unexpanded diameter of the aforementioned expandable cage-type spinal stabilization devices to their expanded diameter can range from about 1:1.2 to 1:4, preferably about 1:1.4 to 1:3.

Of course, depending on the patient's anatomy, larger or smaller or longer or shorter spinal stabilization devices may be utilized, as the dimensions cited above are applicable only to the skeleton of a particular 60 year old male of average size.

The interior of the coil or cyclinder of the spinal stabilization device may be filled or packed with autologous or cadaver bone and, optionally, with bone growth stimulating agents, such as bone morphogenic protein (BMP) to promote the ingrowth of fibrous tissue and encapulation of the spinal stabilization device in the disc. Preferably, however, instead of packing the stabilization device with autologous or cadaver bone, a small amount of the patient's own bone marrow, which contains stem cells, can be extracted (aspirated) from the patient's hip or sternum by a syringe, diluted if necessary, filtered and injected into the spinal stabilization device to accellerate fibrous encapsulation of the device in the disc and the stem cells' repopulation of the nucleus pulposa and the annulus of the disc.

Optionally, the filtered bone marrow may be mixed with a thixotropic material, such as microcrystalline cellulose, and can be injected into the spinal stabilization device before or after its insertion into the tunnel in the disc, forming a very viscous matrix. Alternatively, autologous bone marrow can be mixed with bovine or other collagen or added to one or more pre-formed collagen rods or spacers, which may act as a scaffold on which the stem cells may multiply. The stem cells may cause the nucleus pulposa of the disc to be repopulated, filling at least partially, the tunnel made in the disc, and may repopulate the annulus and closing, at least partially, the opening made in the annulus. Additional stem cells from the patient's blood or other tissue may be collected and added to the bone marrow prior to its injection.

While two or more spinal stabilization devices can be inserted into the disc, preferably only one spinal stabilization device is inserted, creating single pivot points between the device and the vertebra above and below the device. This takes the pressure off the degenerated disc, preserves the spine's mobility and maintains the proper space between the vertebra. If the spinal stabilization device is positioned diagonally (posterior-laterally) between the vertebra, instead of across their length (posterior to anterior) or across their length, laterally, less forward and back rocking and less side to side rolling will occur.

### BRIEF DESCRIPTION OF THE DRAWINGS

FIG. 1 is a external view of the laser energy delivery elements of the present invention, with a partial, cross-sectional view of the side-firing laser device;

FIG. 2 is a partial, cross-sectional, side view of an alternative embodiment of the side-firing portion of the laser device of FIG. 1;

FIG. 3 is an external, side view of a portion of a human pine;

FIG. 4 is an external, side view of the spine, with an expanded partial view illustrating a normal and a degenerated lumbar disc;

FIG. 5(a) is an external, side view of a cylindrical spinal cage of the present invention;

FIG. 5(b) is an expanded, cross-sectional, end view of the ribbon of the coil of FIG. 5(a);

FIG. 5(c) is an external, side view of an alternate embodiment of a cylindrical metal cage of the present invention;

FIG. 5(d) is a cross-sectional, side view of the cylindrical cage of FIG. 5(c);

FIG. 5(e) is an external, side view of another alternate embodiment of the cylindrical cage of the present invention;

FIG. 5(f) is an external, side view of a spherical cage of the present invention;

FIG. 6(a) is a cross-sectional, side view of the nose piece of the present invention;

FIG. 6(b) is an external, distal end view of the nose piece of FIG. 6(a);

FIG. 7(a) is a cross-sectional, side view of the end plate of the present invention;

FIG. 7(b) is an external, proximal end view of the end plate of FIG. 7(a);

FIG. 7(c) is a cross-sectional, side view of the end plate at plane A-A of FIG. 7(a) with the tool of FIG. 8 inserted;

FIG. 8 is a cross-sectional, side view of a tool for use with certain embodiment of the present invention;

FIG. 9 is a cross-sectional, side view of assembled spinal implant device of the present invention;

FIG. 10 is a cross sectional, side view of the spinal stabilization device of the present invention inserted into a spinal disc;

FIG. 11(a) is a cross-sectional, side view of an expandable embodiment of the spinal stabilization device of the present invention;

FIG. 11(b) is a cross-sectional, side view of the expanded spinal stabilization device of FIG. 11(a);

FIG. 11(c) is an external, elevational side view of the expanded device of FIG. 11(b);

FIG. 12(a) is a cross-sectional, side view of a preferred embodiment of the spinal stabilization device of the present invention;

FIG. 12(b) is a cross-sectional, side view of the expanded spinal stabilization device of FIG. 12(a);

FIG. 13(a) is a cross-sectional, side view of an alternate embodiment of the spinal stabilization device of FIG. 12(a);

FIG. 13(b) is a cross-sectional, side view of the expanded spinal stabilization device of FIG. 13(a);

FIG. 14 is an external, top plan view of a precursor of an expandable spinal stabilization device of the present invention;

FIG. 15 is a cross-sectional, side view of the precursor of FIG. 14 formed into the spinal stabilization device of the present invention;

FIG. 16 is a partial, cross-sectional, side view of a two layer spinal stabilization device of the present invention;

FIG. 17 is a simplified diagrammatic view of two unpreferred and one preferred position of the device of the present invention between the vertebra;

FIG. 18 is a cross-sectional, side view of a further embodiment of the spinal stabilization device of the present invention;

FIG. 19 is a cross-sectional, side view of a still further embodiment of the spinal stabilization device of the present invention;

FIG. 20 is a cross-sectional, side view of yet a further embodiment of the spinal stabilization device of the present invention;

FIG. 21 is a cross-sectional, side view of still another embodiment of the spinal stabilization device of the present invention;

FIG. 22 is an end elevational view of the device of FIG. 21;

FIG. 23 is a end elevational view of an alternate spinal stabilization device of the present invention;

FIG. 24 is a cross-sectional, side view of yet a further embodiment of the spinal stabilization device of the present invention;

FIG. 25 is a cross-sectional, side view of still a further embodiment of the spinal stabilization device of the present invention;

FIG. 26 is a cross-sectional, side view of still another embodiment of the spinal stabilization device of the present invention;

FIG. 27 is a cross-sectional, side view of the device of FIG. 26 in its expanded condition; and

FIG. 28 is an end elevational view of yet another embodiment of the spinal stabilization device of the present invention.

### DETAILED DESCRIPTION OF THE INVENTION

To treat a degenerated spinal disc in minimally invasive, outpatient procedure, as described in greater detail above, a guidewire is inserted into the disc, a passageway is created up to the facet bone, an endoscope is inserted and laser energy or mechanical tools are used to remove a small, non-load bearing portion of the facet bone (usually the inferior facet bone, but sometimes the superior facet bone) to create an opening into the foraminal space in the spine, enabling the endoscope to be inserted into the foramen. The surgeon can then see the vertebra, disc and nerves. Graspers, rotating shaver, burr or auger, laser, or RF energy may then be used to create a bore hole or tunnel through the annulus into the nucleus pulposa of the disc. The endoscope is removed, the tunnel into the disc may be widened if necessary, the passageway is further dilated, and a delivery cannula is inserted with its distal end positioned opposite the proximal end of the tunnel in the disc.

A novel spinal stabilization device, comprising a spinal cage that includes a coil or perforated cylinder which may have helical, lateral or longitudinal ridges or threads about its exterior, a bullet-shaped distal end, and an end plate attached to its proximal end, is screwed or tapped into place in the tunnel made in the disc, positioned diagonally and centered in the disc. The bullet shaped distal end of the spinal stabilization device enables it to more easily be inserted into the tunnel in the disc, with less trauma to the disc and opposing vertebra. While two or more of such spinal stabilization devices may be inserted into a disc, preferably only one such device is inserted, creating single pivot points against the vertebra above and below the device. This preserves the spine's mobility, maintains the intervertebral spacing and takes the pressure off the damaged or diseased disc. The cage may be packed or filled with autologous or cadaver bone, which may optionally be fortified with bone growth stimulating materials, such as BMP, to cause fibrous encapsulation of the device in the disc.

Preferably, instead of autologous or cadaver bone, the cage may be filled with a small amount of the patient's bone marrow. As described in co-owned U.S. patent application Ser. No. 09/406,257 (WO 01/20999), which is incorporated herein to the extent applicable, about 3 to 10 ml, preferably about 4 to 7 ml of the patient's own (autologous) bone marrow, which contains stem cells, may be aspirated by syringe from the patient's hip, sternum, femur or other large bone, diluted with phosphate buffered saline if necessary, passed through one or more 100 to 400 micron filters and injected into the spinal stabilization device through an injection port in the end plate (or between the slats of a preferred embodiment) before or after the spinal stabilization device has been tapped or screwed into place in the tunnel in the disc. Optionally, stem cells isolated from the patient's blood or other tissue may be added to the bone marrow to increase the population of injected stem cells, prior to injection of the bone marrow into the spinal stabilization device.

The stem cells may differentiate into nucleus pulposa, filling, at least partially, the tunnel made in the disc. The stem cells may also differentiate into annulus cells and repair, at least partially, the opening made in the annulus of the disc. Optionally, prior to injection, as described in the aforementioned patent application, the bone marrow/stem cell mixture may be mixed with a thixotropic material, such as microcrytalline cellulose or carboxymethyl cellulose sodium, sold as AVICEL® by FMC Corporation (Chicago, Ill.), or a hydrocolloid material such as polyvinylpyrrolidone. Thixotropic mixtures are fluid under pressure, such as during injection through a syringe. However, when the injection pressure is removed, the mixture becomes very viscous, preventing the bone marrow/stem cell mixture from leaking out of the spinal stabilization device. Alternatively, bovine or other collagen, in a viscous preparation or formed into rods or plugs, may be used with autologous bone marrow, as described above.

Mechanical tools, such as a rasp or a rotating shaver, burr or auger, or surgical tools, as known in the art, may be used to remove a portion of the facet bone to create an opening into the foraminal space. However, extensive irrigation is required to remove bone fragments, shavings and debris, the success of which is not assured. Preferably, laser energy is utilized to vaporize a portion of the facet bone to make an opening into the foraminal space, as it is able to do so without creating bone fragments, shavings or other debris, and without thermally damaging or weakening the facet bone.

While RF energy may be used to shrink the nucleus pulposa and annulus of the disc, the shrinkage effect of RF energy is superficial and not as long lasting as that of laser energy. Holmium laser energy is preferred for shrinking the nucleus pulposa and annulus of the disc and vaporizing extruded disc material and other tissues, as it does so efficiently, penetrating precisely 0.4 mm into the tissue, without creating charring, and with long lasting results.

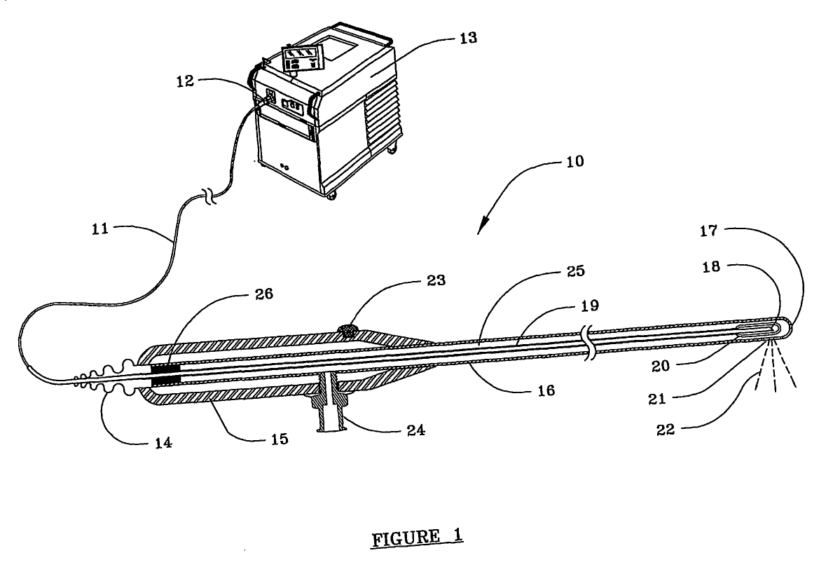

As seen in FIG. 1, laser energy delivery system 10 includes optical fiber 11, whose proximal end is encased within connector 12 and is optically coupled to a source of laser energy 13. Optical fiber 11 is affixed within and extends through flexible plastic or rubber strain-relief 14, handpiece 15 and hollow metal tube 16, which is preferably made of medical grade stainless steel, and whose distal end 17 is blunt ended to be less traumatic to tissue. The distal end face 18 of optical fiber 11 is beveled at an angle of about 30 degrees to 50 degrees from the longitudinal axis of the optical fiber 11, preferably at an angle of about 35 to 45 degrees, most preferably at an angle of about 39 to 40 degrees, and terminates proximal to the distal end 17 of tube 16. The buffer coating and vinyl cladding 19 of optical fiber 11 have been removed from the distal end portion of optical fiber 11 by means known in the art, and quartz or fused silica capillary tube 20, whose distal end has been closed by thermal fusing, is attached by an adhesive or thermal fusing to the proximal end of the bare distal end portion of optical fiber 11.

Capillary tube 20 creates and maintains an air interface at the beveled distal end face 18 of optical fiber 11. When laser energy is transmitted from laser 13 through optical fiber 11, the laser energy is directed by total internal reflection from the beveled distal end face 18 of optical fiber 11 at an angle of about 50 to 110 degrees from the axis of optical fiber 11, preferably at an angle of about 70 to 90 degrees, through port 21 in metal tube 16, as shown by arrows 22. Button 23 on handpiece 15 is positioned opposite to the direction in which laser energy is emitted and enables the surgeon, by tactile feel or visual observation, to ascertain the direction in which the laser energy will be emitted.

Luer lock 24 enables a small amount of fluid from an external source to be infused into fluid channel 25 of tube 16 to cool and flush debris from the quartz or fused silica capillary tube 20 and cool the metal tube 16. Alternatively, suction may be applied to luer lock 24 to draw hot gasses from the vaporization of tissue into port 21 and channel 25 of tube 16, which can be captured in a vacuum collection bottle (not shown), as known in the art. Gasket 26 prevents fluid from exiting the proximal end of handpiece 15.

Alternatively, a second fluid channel (not shown) may be provided in metal tube 16, and a second luer lock (not shown) may be provided in handpiece 15, communicating with said second fluid channel. Fluid may be infused through the first channel to flush debris from capillary tube 20 and cool metal tube 16 after lasing, and a vacuum may be applied to the second channel to remove excess fluid and hot gasses from the vaporization of tissue during lasing. Two channels permit both functions to be performed without having to disconnect a vacuum source and connect a liquid source. Two channels also permit the concomitant infusion of fluid through one channel and suction of hot gasses and excess fluid through the other channel. Such channels are described in co-owned U.S. patent application Ser. No. 10/127,382, which is fully incorporated herein by reference.

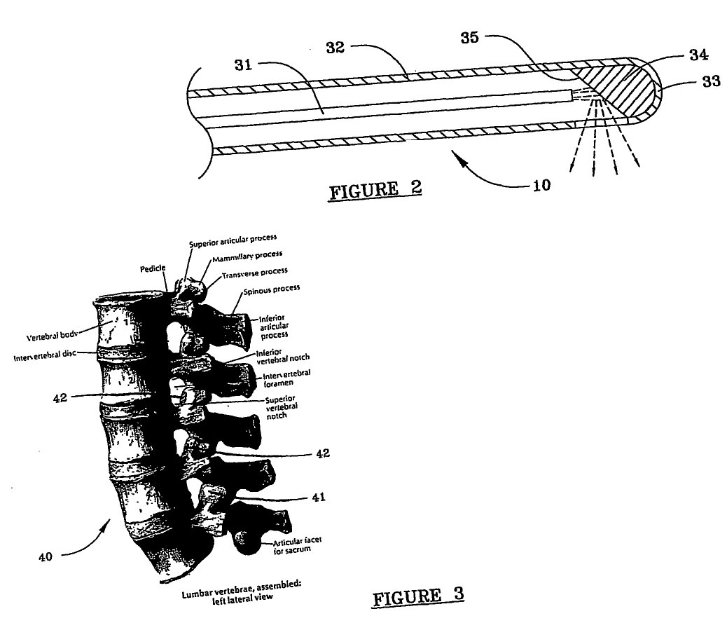

FIG. 2 illustrates an alternate embodiment of the laser energy emitting portion of the device of the present invention. Energy emitting device 30 consists of optical fiber 31 which is disposed within hollow metal tube 32, whose distal end 33 is blunt ended. The distal end 33 of tube 32 has been filled with a metal plug 34, whose proximal end surface 35 has been beveled at an angle of about 30 degrees to 60 degrees from the axis of tube 32, preferably at about 40 degrees to 50 degrees, and most preferably at about 45 degrees.

Plug 34 can be made of a reflective material, such as gold, silver, copper, or a dieletric. Silver is preferred, as it reflects Holmium laser energy about as effectively as gold or copper, is substantially less expensive than gold and is more durable than copper. Alternatively, plug 34 can consist of a metal, such as stainless steel, to whose beveled end face 35 a sheet or layer of gold, silver, copper or a dielectric material (not shown) may be fixedly attached. Laser energy transmitted through optical fiber 31, is reflected from the reflective, beveled end surface 35 of plug 34 and exits port 36 of tube 32 at an angle of about 80 to 90 degrees from the axis of optical fiber 31, as shown by arrows 37.

Devices such as those shown in FIGS. 1 and 2 above are more fully described in U.S. Pat. Nos. 5,242,437, 5,380,317 and 5,649,924, and their method of use is described in U.S. Pat. No. 5,437,660, all of which are fully incorporated herein by reference.

FIG. 3 is a side view of a portion of human spine 40, showing inferior facet bone 41 and the area 42 of interior facet bone 41 to be removed to create an opening into the inter-vertebral foramen.

FIG. 4 shows a portion of human spinal column 50. As seen in the enlarged portion of spinal column 50, disc 51 is normal whereas disc 52 is degenerated, having been damaged, diseased or dehydrated (due to old age), reducing the space between upper vertebra 53 and lower vertebra 54.

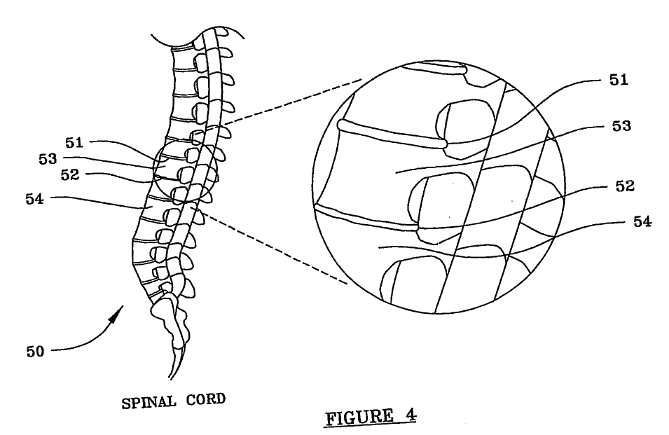

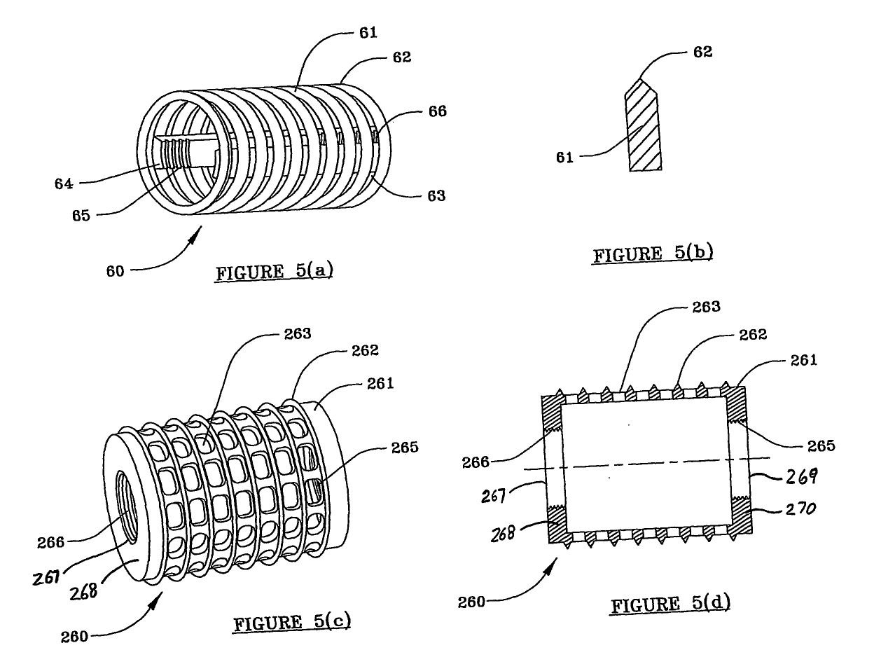

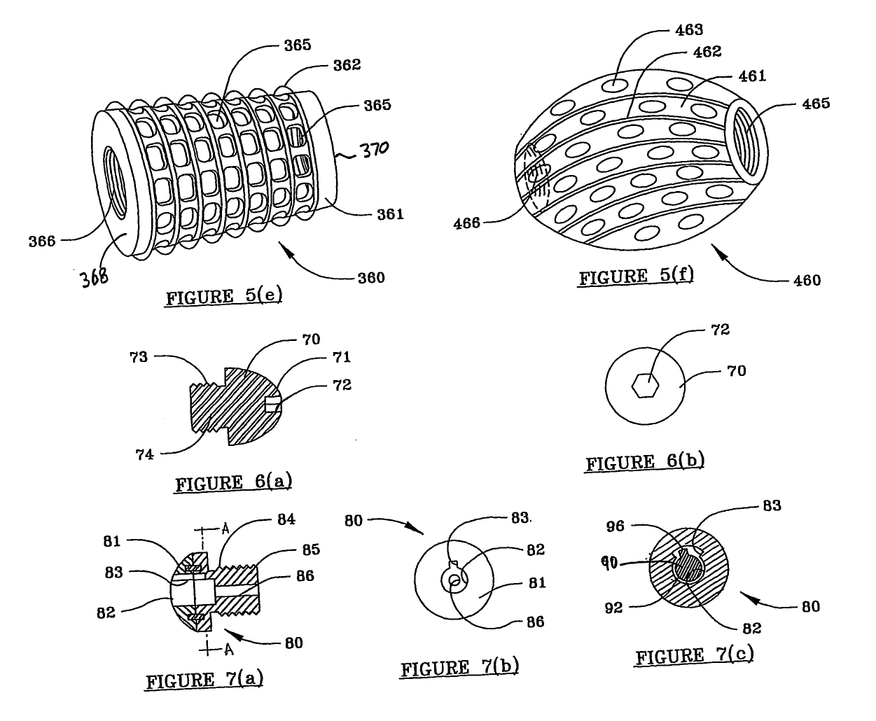

FIG. 5(a) illustrates a first embodiment of a cage 60 of the spinal stabilization device of the present invention. Cage 60 comprises a helical metal coil 61, which may be made of stainless steel or other metal, but is preferably made of titanium or a nickel-titanium alloy. Alternatively, cage 60 can also be made of high density polyethylene or a carbon wire reinforced, strong resilient plastic. Coil 61 can be formed from a metal ribbon, whose exterior edges 62 are beveled into a sharp point and function as threads, as shown, or can be formed from a metal ribbon, wire or rod of any other desired cross section, with at least small spaces 63 between each of the individual coils 61 of cage 60.

Two or more longitudinal bars 64 are attached by crimping, welding or other means known in the art to the interior surface of coils 61. Bars 64 are positioned opposite each other in a generally co-planar relationship and have helical threads 65 and 66 on at least a portion of the interior of their distal and proximal ends, respectively. Threads 65 and 66 on bars 64 allow the threaded, proximal end portion of the nose piece of the spinal stabilization device described in FIG. 6(a)-(b) below and the threaded, distal end portion of the end plate of the spinal stabilization device described in FIG. 7(a)-(c) below, respectively, to be threadingly secured into the distal and proximal ends, respectively, of coil 61.

The sharpened exterior edges 62 of coil 61 function as helical threads and enable the fully assembled spinal stabilization device shown in FIG. 9 to be screwed into the tunnel earlier created in the disc and prevent movement or dislodgment of coil 61 from the disc. The threads should be made with about 2 to 12 turns per centimeter of length of the cage, preferably about 3 to 8 turns per centimeter of length.

FIG. 5(b) is an enlarged, cross-sectional, end view of ribbon 61 used to form coil 60 of FIG. 5(a), with exterior edge 62 beveled into a sharp point to form a thread. Ribbon 61 may have any other desirable cross-section.

An alternative embodiment of the cage 60 shown in FIG. 5(a) is shown in FIG. 5(c). In this embodiment, cage 260 consists of hollow metal cylinder 261, which may be made of stainless steel or other metal, but is preferably made of titanium or a nickel-titanium alloy or a strong, resilient material such as a carbon fiber reinforced plastic, or a very high density plastic, such as high density polyethylene. Cylinder 261 has helical threads 262 formed on its exterior surface. Ports 263 are formed in the surface of the cylinder 261 and extend in spaced-apart relationship around the circumference thereof in the space defined between each of the threads 262 to permit ingrowth of fibrous tissue or egress of stem cells, as described above. Helical threads 266 are also formed on the interior surface of an opening 267 defined in a proximal radial end wall 268 of cylinder 261 and helical threads 265 are formed on the interior surface of an opening 269 defined in a distal radial end wall 270 of cylinder 261. Exterior cylinder threads 262 enable cylinder 261 to be screwed into place in the tunnel earlier created in the disc and prevent movement within or dislodgment of cylinder 261 from the disc, with the same number of turns per centimeter of length described above. Threads 265 and 266 enable the threaded nose piece of the spinal stabilization device described in FIGS. 6(a)-(b) below and the threaded end plate of the spinal stabilization device described in FIGS. 7(a-(c) below, respectively, to be screwed into and affixed within the distal and proximal ends, respectively, of cylinder 261.

FIG. 5(d) is a cross-sectional, side view of cage 260 of FIG. 5(c), with threads 265 within the interior surface of the distal radial end wall 270 of cage 260 and threads 266 within the interior surface of the proximal radial end wall 268 of cage 260. Ports 263 enable fibrous tissue ingrowth or stem cell egress, as described above. Ports 263 should constitute at least 15% of the exterior surface of the cage, preferably about 20% to 35%.

Another cage embodiment is shown in FIG. 5(e) which discloses a cage 360 similar in a structure to cage 260 except that it includes a tapered or cone shaped cyclinder 361, with helical threads 362 spaced about its exterior surface, ports 363 for bone ingrowth or stem cell egress, helical threads 365 within the interior of its distal radial end wall 370 and threads 366 within the interior of its proximal radial end wall 368. Cyclinder 361 can alternatively be made with its larger diameter at its distal end and its smaller diameter at its proximal end. Selecting and inserting the proper tapered cyclinder 361 enables the natural lordotic space between the vertebra (not shown) to be maintained.

In alternate embodiments of the devices of FIGS. 5(c) and (d), the exterior threads 262 and 362 of cylinders 260 and 360 respectively may be longitudinal (not shown), enabling cylinders 260 and 360 to be tapped or pressure forced into place in the tunnel earlier made in the disc.

Yet another alternate cage embodiment of cage 60 is seen in FIG. 5(f), in which cage 460 consists of a hollow sphere 461, with ridges 462 extending longitudinally along the exterior of the sphere in spaced-apart, circumferential relationship and a series of rows of generally oval-shaped ports 463 defined in the exterior surface of the sphere 461 and extending in spaced-apart, longitudinal relationship in the space defined between each of the ridges 462. Sphere 461 provides maximum mobility of the spine by creating very small, single pivot points between sphere 461 and the vertebra (not shown) above and below sphere 461. When sphere 461 is tapped or pressure forced into the tunnel earlier made in the disc (not shown), ridges 462 prevent movement within and dislodgement of sphere 461 from the disc. Helical threads 465 extend within at least a portion of the interior surface of the distal end of sphere 461, and threads 466 extend within at least a portion of the interior surface of the proximal end of sphere 461, enabling the threaded nose piece described in FIGS. 6(a) and (b) and the threaded end plate described in FIGS. 7(a)-(c) to be screwed into and fixably attached within the distal and proximal ends, respectively, of hollow sphere 461. External ridges 462 can, alternatively, be spaced-apart threads extending helically around the exterior of sphere 461.

For example, for use in a lumbar disc of a particular, average sized, 60 year old male, cages 60, 260, 360 and 460 of FIGS. 5(a) and 5(c)-(f) can have an outer diameter, including their threads or ridges, of about 6 to 14 mm, preferably about 8 to 12 mm in diameter. Likewise, for use in a thoracic disc, said cages 60, 260, 360 and 460 including their threads or ridges, can have an outside diameter of about 4 to 10 mm in diameter, preferably about 5 to 8 mm in diameter, and for use in a cervical disc, said cages 60, 260, 360 and 460 including their threads or ridges, can have an outer diameter, of about 2 to 7 mm, preferably about 3 to 6 mm. However, as mentioned above, if the surgeon wishes to promote fusion and bone growth into the device, larger diameter cages 60, 260, 360 and 460 may be packed with bone and inserted, requiring the removal of a portion of the end plates and cancellous bone of the vertebra above and below the degenerated disc. The outside diameter of cages 60, 260, 360 and 460 is determined by the surgeon, based upon x-rays of the patient's spine in the area to be treated, his observation of the patient's anatomy and his medical judgement.

The cages of the present invention can also be oval in cross-section, oval with flattened surfaces at 12 and 6 o'clock or 3 and 9 o'clock positions, or of any other desired shape, with helical, lateral or longitudinal threads or ridges about their exterior surface depending upon particular uses.

As seen in FIG. 6(a), nose piece 70 has a bullet shaped distal end or head 71. Nose piece 70 may be made of stainless steel or other metal, but is preferably made of titanium, a nickel-titanium alloy, a high density plastic such as high density polyethylene, a carbon fiber reinforced plastic, or a ceramic (polychrystalline alumina). Nose piece 70 contains a centrally located recess 72 extending into the interior of distal end 71 and helical threads 73 formed about an abutment, finger or screw 74 formed on the proximal end of nose piece 70 and extending outwardly from the rear end of the bullet shaped distal end 71. Helical threads 73 on finger 74 enable nose piece 70 to be screwed into and affixed within the threaded distal end of cages 60, 260, 360 and 460 of FIGS. 5(a) and 5(c)-(f).

As shown in FIG. 7(a), end plate 80 has a rounded proximal end or head 81, into which a centrally located channel 82 and keyway 83 extend. End plate 80 has helical threads 84 formed about abutment, finger or screw 85 extending and depending outwardly from the head 81. Injection port 86 extends centrally through the finger 85 of end plate 80. End plate 80 may also have a flat proximal end face or any other desired shape.

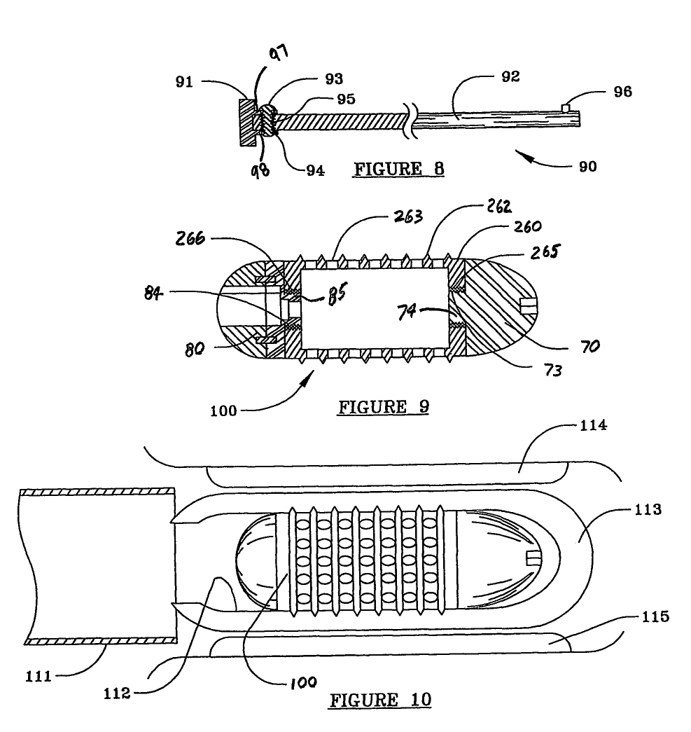

Channel 82 and keyway 83 of end plate 80 enable the shaft of an insertion or screwing device, with a pin or key on its distal end, as described in FIG. 8 below, to be inserted into channel 82 and keyway 83, respectively, and, when turned clockwise or counterclockwise, to rotate end plate 80, both to screw end plate 80 into the proximal end of one of the cages described above and to screw the entire, assembled spinal stabilization device shown in FIG. 9 below into the channel earlier made into the disc, as illustrated in FIG. 10 below. Such devices must be sufficiently strong to endure 70 to 110 ft.-lbs. of torque when being screwed into the tunnel in the disc.

As seen in FIG. 7(b), end plate 80 has channel 82 in its proximal end to accommodate the shaft of the screwing or insertion device shown in FIG. 8 below, and keyway 83 to accommodate the pin or key on the distal end of the shaft of said screwing or insertion device. While channel 82 and keyway 83 terminate within the body of the head 81 of the end plate 80, as shown in FIG. 7(a), injection port 86 is in fluid flow communication with the channel 82 and extends completely through end plate 80 and the finger 84 so as to enable bone growth stimulating agents or bone marrow and/or stem cells to be injected through the channel 82 and the port 86 and into the cages shown in FIGS. 5(a) and 5(c)-(f).

FIG. 7(c) shows end plate 80, channel 82 and expanded keyway 83. Keyway 83 has been widened to about 20 to 90 degrees, preferably about 40 to 60 degrees, to each of the left and right of the vertical axis of end plate 80. As shown, shaft 92 and key 96 of the screwing device 90 shown in FIG. 8 are disposed within channel 82 and keyway 83 of end plate 80, respectively. Widened keyway 83 enables the pin or key 96 of the screwing device shown in FIG. 8 below, when turned to the right or left of the vertical axis, to be held in place within expanded keyway 83. When the screwing device of FIG. 8 below is rotated clockwise or counter-clockwise, end plate 80 and its attached cage and nose piece (not shown) are screwed into or out of, respectively, the tunnel in the disc (not shown). When the pin or key of said screwing device is re-aligned with keyway 83, said screwing device can be withdrawn from end plate 80, leaving the spinal stabilization device in place in the tunnel in the disc. Likewise, the shaft and key or pin of the insertion device can be engaged within channel 82 and keyway 83 and used to tap or force end plate 80 and its attached cage and nose piece (not shown) into place in the tunnel in the disc (not shown).

FIG. 8 illustrates insertion tool 90, which includes a handle or head 91, which is fixedly attached to shaft 92 by a screw 93 which extends through a pair of fingers 97 and 98 which depend generally normally outwardly from the head 91 and whose threads 94 engage threads 95 formed on the interior of a bore defined and extending through the shaft 92 adjacent the proximal end thereof. The proximal end of shaft 92 is wedged between the fingers 97 and 98 and the screw 93 extends throught the fingers 97 and 98 and the shaft 92. Alternatively, handle 91 may be affixed to shaft 92 by welding or other means, as known in the art. Key 96 at the distal end of shaft 92 is sized to slidably fit within the keyway 83 of end plate 80 shown in FIGS. 7(a)-(c).

FIG. 9 illustrates one embodiment of the fully assembled spinal stabilization device 100 of the present invention. As can be seen, bullet shaped nose piece 70 has been screwed or threadingly secured into the distal end of the cylinder 260, Threads 73 on finger 74 of nose piece 70 engage internal threads 265 within the distal end of cylinder 260. While not separately shown, nose piece 70 can likewise be screwed into coil 61 of cage 60 of FIG. 5(a) or any of the other cages shown in FIGS. 5(e)-(f). Threads 84 on the finger 85 of end plate 80 engage threads 266 within the proximal end of cylinder 260, enabling end plate 80 to be fixably attached to the proximal end of cylinder 260. Helical threads 262 extend about the exterior of cylinder 260, enabling the device 100 to be screwed into the disc (not shown). Ports 263 enable bone ingrowth or stem cell egress, as described above.

Alternatively, nose piece 70 and end plate 80 can be pressure-fitted within cylinder 260, attached by an adhesive, welded thereto or otherwise attached to cylinder 260 by means known in the art. In an alternative embodiment, instead of spinal implant device 100 consisting of a separate nose piece 70, cylinder 260 and end plate 80, the entire spinal stabilization device can be forged or milled from a single piece of metal or ceramic, or formed of a very high density plastic.

For example, for use in a degenerated lumbar disc of a particular, average sized, 60-year old male, fully assembled spinal stabilization device 100 may be about 20 to 30 mm in length, preferably about 23 to 27 nm long. Similarly, for use in a degenerated lower thoracic disc, spinal stabilization device 100 can be about 17 to 25 mm long, preferably about 19 to 23 mm long, or for use in a degenerated upper thoracic disc, device 100 can be about 15 to 30 mm long, preferably about 12 to 19 mm in length. Likewise, for use in a degenerated lower cervical disc, spinal stabilization device 100 can be about 8 to 14 mm in length, preferably about 10 to 12 mm long, or for use in a degenerated upper cervical disc, device 100 can be about 5 to 10 mm in length, preferably about 6 to 9 mm long.

FIG. 10 shows assembled spinal stabilization device 100 inserted through a delivery cannula 111 into a tunnel 112 in spinal disc 113. Vertebra 114 above and vertebra 115 below disc 113 are properly spaced apart by spinal stabilization device 110 and the remainder of disc 113.

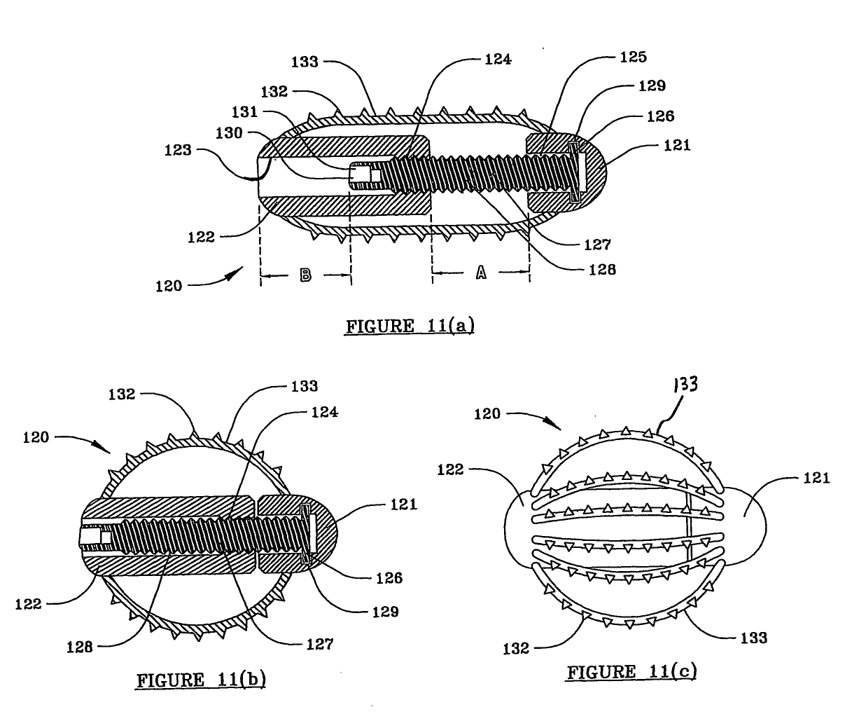

In a preferred embodiment, as seen in FIG. 11(a), a mechanically expandable spinal stabilization device 120 is made of metal, preferably titanium or a nickel-titanium alloy, which has great mechanical strength, or a very strong, resilient material, such as carbon fiber reinforced plastic or high density polyethylene, as the pressure of the vertebra on the spine can reach up to 800 pounds or more per square inch. Distal end nose piece 121 of spinal stabilization device 120 is rounded or bullet shaped. Proximal end piece or plate 122 of spinal stabilization device 120 also has a rounded proximal end, although it can have a flat proximal end face or any other desired shape. A bore 123 is defined in and extends centrally longitudinally through the end plate 122 and terminates within the body of the plate 122 into a threaded interior distal radial end wall 124. Distal end nose piece 121 defines an interior channel or cavity 125 and a circumferential recess 126 extending into the body of piece 121 from the channel 125.

Elongate bolt 127 extends longitudinally through the bore 123 in piece 122 and the channel 125 in piece 121 and has threads 128 about its exterior adapted to allow the threading engagement between the bolt 127 and the piece 122 in the region of the threaded radial end wall 124. The bolt 127 also defines a distal radially outwardly extending flange 129 adapted to be lodged in the recess 126 defined in the interior of the distal end piece 121. Bolt 127 defines a channel 130 and keyway 131 formed in a proximal end thereof, similar to the channel and keyway described in FIGS. 7(a) and (b) in connection with end plate 80. Keyway 131 has an expanded interior similar to that shown above in FIG. 7(c). Channel 130 and keyway 131 enable the shaft and pin of the insertion tool shown in FIG. 8, respectively, to be inserted and used to advance spinal stabilization device 120 into the channel earlier made in the disc (not shown). Points or spikes 132 extending outwardly from the exterior surface of slats 133 prevent movement or dislodgement of device 120 after its insertion into a disc. Preferably, spikes 132 are positioned to form interrupted helical threads about the exterior of device 120, enabling it to be screwed into place in the tunnel earlier made in the disc.

Slats 133, which are preferably made of elongate strips of titanium or a nickel-titanium alloy, are attached to and extend between the proximal and distal end pieces 121 and 122, respectively of device 120 by welding, pins, screws, crimping or other means known in the art. A plurality of the slats 133 extend longitudinally between the proximal end piece 122 and the distal end piece 121 in spaced-apart and circumferential relationship around the pieces 121 and 122 so as to define a bird-like shaped cage.

As shown in FIG. 11(b), when the shaft and key of the insertion tool shown in FIG. 8 are inserted into channel 130 and keyway 131 of bolt 127, respectively, and the handle of the insertion tool is rotated, the key of the insertion tool engages the left or right face of the expanded portion of keyway 131, threading proximal end piece 122 along threaded bolt 127 in the direction of the distal end piece 121. As end piece 122 advances along threaded bolt 127, slats 133 expand or bulge outwardly. When proximal end piece 122 has traveled rearwardly a distance A, the distal end of proximal end piece 122 is disposed adjacent the proximal end of distal end piece 121, and slats 133 have been fully extended. Likewise, when bolt 127 has advanced forwardly a distance B, the proximal end of bolt 127 is disposed generally co-planarly with the proximal end face of proximal end piece 122. Notably, when bolt 127 is rotated, proximal end piece 122 moves along bolt 127, and distal end piece 121 remains in its desired position in the tunnel in the disc.

As shown in FIG. 11(c), spinal stabilization device 120 has a total of ten slats 133, of which six slats 133 are visible in this external view of device 120. However, device 120 may contain any number of slats, wider or narrower than those shown, to resist the pressure of the vertebra on device 120.

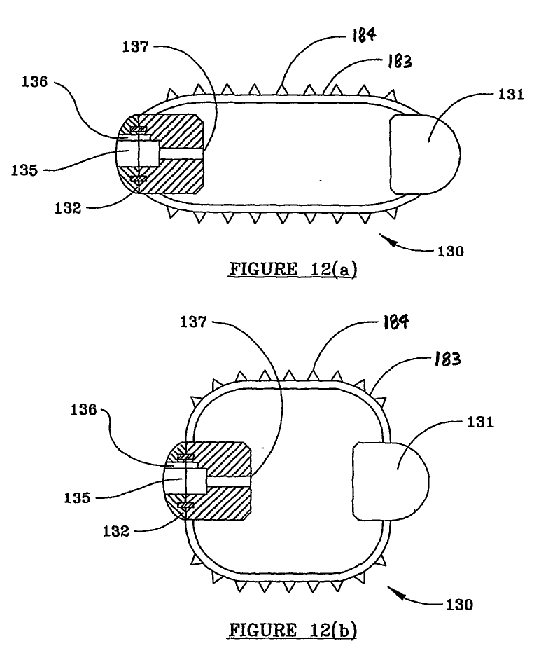

FIG. 12(a) illustrates a more preferred embodiment of the present invention. Spinal stabilization device 130 includes a bullet-shaped distal end nose piece or head 131 and a rounded proximal end piece or plate 132 similar in configuration to end plate 80 except that it does not include a threaded outer surface. Slats 183 are similar to slats 133 described above in connection with the embodiment of FIGS. 11(a)-(c). Slats 183 have points or spikes 184 on their exterior surface, which may function as interrupted threads and facilitate the screwing of the device 130 into the tunnel in the disc and, when properly positioned, prevent movement of stabilization device 130 within the disc and maintain its position between the vertebra. Proximal end piece or plate 132 is similar to end plate 80 and defines an interior channel 135, a keyway 136 and an elongate interior injection port 137 which extends from the distal end of channel 135 through the distal end of piece 132 and is in fluid communication with the channel 135 and the interior of device 130. As described above in connection with the plate 80, channel 135 and keyway 136 may be engaged by the shaft and key of the insertion tool shown in FIG. 8 and enable spinal stabilization device 130 to be inserted into and properly positioned within the tunnel in the disc (not shown), as shown in FIG. 10 above. Insertion port 137 enables bone growth-stimulating materials or bone marrow/stem cells to be injected into device 130 before or after its insertion into the tunnel in the disc as described earlier with respect to the port 87 of plate 80.

The distal and proximal ends of the slats 183 are attached to distal end piece 131 and proximal end piece 132, respectively, by welding, pins, screws, crimping or other means known in the art. Spikes 184 may also be positioned radially to act as interrupted, helical threads to enable stabilization device 130 to more easily be screwed into the tunnel earlier made in the disc.

In this embodiment, slats 183 are made of a superelastic shaped-memory, nickel titanium alloy known as nitinol. When slats 183 are below their transition temperature, for example about 65° to 90° F. (about 8° to 26° C.), slats 183 have the shape shown in FIG. 12(a). Prior to insertion into the disc, stabilization device 130 may be stored in a refrigerator or immersed in cold (refrigerated) sterile water or saline at a temperature of about 40° to 45° F. (about 2° to 5° C.).

As seen in FIG. 12(b), when spinal stabilization device 130 has been inserted into a disc and reaches its transition temperature, for example, about 65° to 90° F. (about 8° to 26° C.), slats 183 resume their thermally programmed shape, expanding and bulging stabilization device 130 to a cylindrical shape, as shown, with a desired pre-set diameter. The surgeon will ascertain the spacing desired between the vertebra by x-ray and select a stabilization device with the proper expanded diameter to insert. Stabilization device 130 can have any number of wide or narrow slats 183, provided they have sufficient strength to resist the force of the vertebra on the slats 183 and the disc.

While slats 183 of the stabilization device 130 shown in FIG. 12(b) have been thermally programmed to create, when heated to their transition temperature, a substantially spherical shape, slats 183 can be thermally programmed to create, when heated above their transition temperature, a cylinder, a tapered cylinder, as shown in FIGS. 5(a) and (c)-(f), an ovoid shape or any other desired shape.

Expandable spinal stabilization devices 120 and 130 of FIGS. 11(a) and 12(a) can be made with a smaller outside diameter than the devices of FIG. 5(a) or (c)-(f). Reducing the diameter of the spinal stabilization devices 120 or 130 enables a smaller diameter delivery cannula to be used and a smaller tunnel to be made in the disc, reducing the trauma to the patient during the insertion process. Also, spinal stabilization device 130 of FIG. 12(a) can be held in its desired position in the tunnel in the disc until it reaches its transition temperature, when it expands almost instantly to its desired shape. If it is desired to re-position spinal stabilization device 130 or remove stabilization device 130 from the channel in the disc, for example, to replace it with a larger or smaller diameter device, cold, sterile water or saline can be infused into the tunnel in the disc, cooling stabilization device 130 below its transition temperature and causing it to return to its smaller diameter shape for re-positioning or removal.

The expanded diameter of the aforementioned mechanically expanded or superelastic, shaped-memory metal spinal stabilization devices can be about 120% to 400%, preferably about 140% to 300% of their unexpanded diameter.

As seen in FIG. 13(a), another stabilization device embodiment 140 consists of distal end nose piece or head 141, proximal end piece or plate 142 and outer and inner slats 143 and 144, respectively, which are made of a superelastic, shaped memory metal, such as nitinol. Outer slats 143 and inner slats 144 are attached to distal and proximal end pieces 141 and 142, respectively, by screws, pins, crimping, welding or any other means known in the art. Outer slats 143 contain points or spikes 145 on their outer surface, which function as described above. The plate 142 is similar in structure to the plate 132 which, in turn, is similar in structure to the plate 80. The head 141 is similar to the head 131. The inner slats 144 extend between the radial proximal end faces of the pieces 141 and 142 respectively while the outer slats 143 define a bird-like cage, surround the slats 143, and extend between the outer circumferential distal end surfaces of the pieces 141 and 142 respectively.

As shown in FIG. 13(b), when heated to their transition temperature, outer slats 143 assume their pre-programmed, expanded, cylindrical shape, and inner slats 144 contract into accordion shaped slats 144 defining one or more flanges 146. The advantage of this embodiment is greater resistance to the pressure of the vertebra on device 140 provided to outer slats 143 by the flanges 146 of inner slats 144. While inner slats 144 have been thermally programmed to form two flanges, as seen in FIG. 12(b), slats 144 can be thermally programmed to form one, three or more such flanges 146. Likewise, while device 140 is shown as including—outer and inner slats 143 and 144, it is understood that the device 140 can be made with any number of pairs of outer and inner slats 143 and 144.

As shown in FIG. 14, the spinal stabilization devices described in FIGS. 11(a) and 12(a) can be made from a single, flat sheet of metal 150, preferably a superelastic, shaped-memory nickel-titanium alloy, such as nitinol, defining cut-out slots 151 extending in spaced-apart and parallel relationship between the top and bottom edges of the sheet 150. Elongate bars or slats 152 are defined between the slats 151, which optionally may have points or spikes 153 extending in spaced-apart relationship along the surface of the slats 152, which function as described above, or may have a smooth or rough textured exterior (not shown).

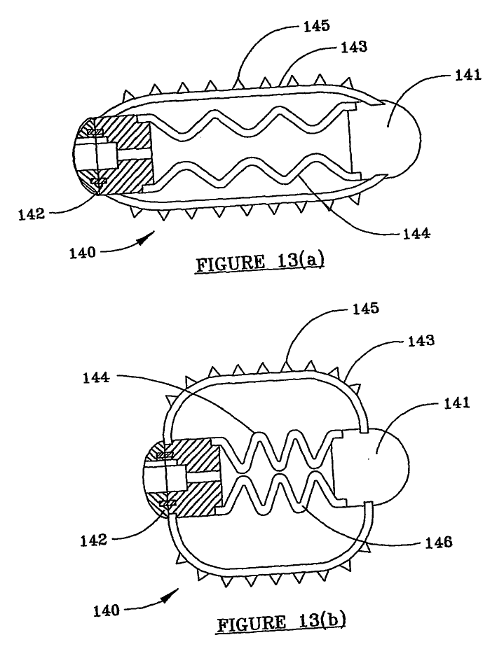

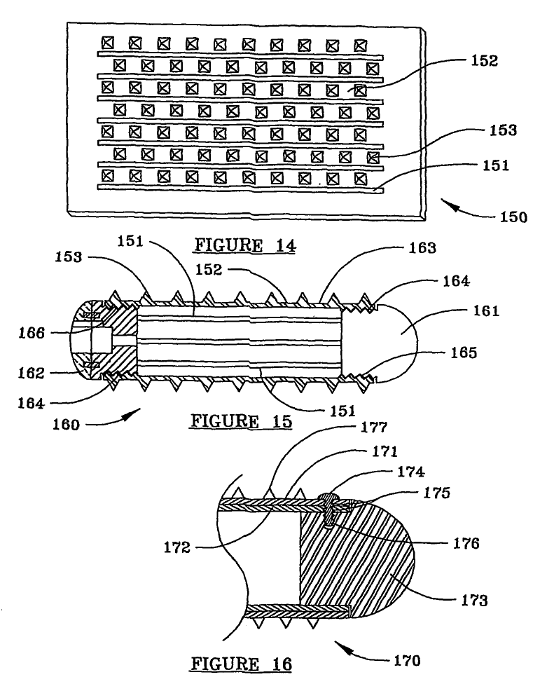

As illustrated in FIG. 15, yet another spinal stabilization device embodiment 160 consists of distal end nose piece or head 161 and proximal end piece or plate 162 similar in structure to the head 70 and plate 80, respectively. The flat metal sheet 150 shown in FIG. 14 has been formed into a tube 163. Helical threads 164 have been formed on the interior surface of the tube 163 adjacent the proximal and distal ends thereof to cooperate and receive threads 165 and 166 formed about the exterior of end pieces 161 and 162 respectively in a manner similar to that described in connection with the FIG. 9 embodiment. Stabilization device 160, when heated above its transition temperature, can expand as described above into the shapes shown in FIGS. 12(b) and 13(b) or any other desired shape. Points or spikes 153 on the exterior of device 160 function as aforesaid.

As shown in FIG. 16, two separate, flat sheets of superelastic, shape-memory metal 150, such as nitinol, with cut outs or slots 151 to define slats 152, as shown and described in FIG. 14, can be formed into a two layer spinal stabilization device 170. Two layer device 170 consists of inner tube 172, outer tube 171 surrounding and abutting the inner tube 172, distal end nose piece 173 and a proximal end piece or plate (not shown), constructed and structured as shown in FIG. 15 above. Screw 174 extends through threaded apertures 175 defined in tubes 171 and 172, respectively, and a threaded aperture 176 defined in the surface of the distal end piece 173 for connecting the tubes to the piece 173. A similar screw (not shown) connects the tubes to the proximal end piece (not shown). Alternatively, tubes 171 and 172 may be welded or crimped together and welded or crimped to distal end piece 171 and proximal end piece (not shown). Tubes 171 and 172 may alternatively be attached together and to the end pieces by any other means known in the art. Points or spikes 177 on the exterior of device 170 function as described above.

When two layer spinal stabilization device 170 is heated to its transition temperature, tubes 171 and 172 can form slats with the shapes shown in FIGS. 11(b) and (c), 12(b), 13(b) or any other desired shape.

While the expandable spinal stabilization devices of FIGS. 11-16 are shown with ridges or spikes on their external surface, which may function as interrupted helical threads, the exterior surfaces of these devices may be smooth or rough finished or textured.

Tapered spinal stabilization devices to fit the lordotic space between the vertebra can be sized, before and after expansion, as described above, but with the narrower end about 10% to 18% less in diameter than the diameter of the devices shown above, preferably about 12% to 15% less in diameter.

All or part of the spinal stabilization devices described above may also be made of porous titanium, with pores of 75-300 microns in diameter. Hydroxyapatite may also be applied to all or part of the above described spinal stabilization devices via an electro-chemical process, as known in the art. Doing so will promote ingrowth of bone or bone attachment to the spinal stabilization device.

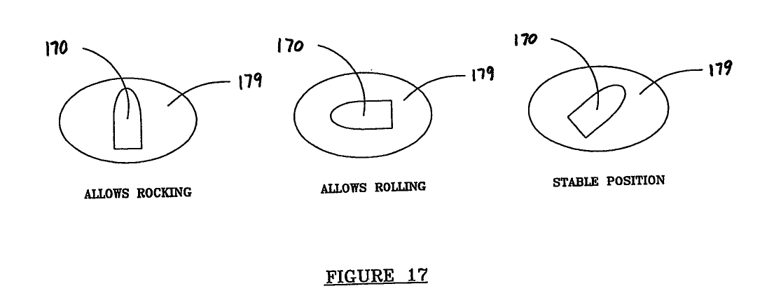

As illustrated in FIG. 17, positioning spinal stabilization device 170 laterally or longitudinally with respect to the vertebral end plates 179 subjects the spine to excessive rolling or rocking. Positioning spinal stabilization device 170 diagonally across vertebral end plates 179 provides greater stability to the spine and is preferred. This position favorably coincides with the postero-lateral approach to the foraminal space, so positioning the device properly is simplified.

As illustrated in FIG. 18, a yet further spinal stabilization device embodiment 180 consists of a solid generally cyclindrical elongate body 181, with a rounded or bullet-shaped distal end face or nose 182 to distract (spread-apart) the vertebra and a flat proximal face radial end 183 with rounded peripheral edges 184. The stabilization device 180 can be made of a metal such as titanium, or a material such as high density polyethylene, or polyethyl ether ketone (PEEK), or a carbon fiber composite. Carbon fiber composites can be made with a modulus of elasticity similar to that of the vertebral bone, whereas titanium has a modulus of elasticity one third of that of the bone (three times the stiffness of bone).

Stabilization device 180 may have a smooth exterior and may be tapped into place in a tunnel created in a degenerated disc (not shown). Optionally, however, to enable it to be threaded into a tunnel created in a degenerated disc (not shown) and to help anchor it in place, stabilization device 180 can be made with helical external threads 185 which extend along the length of and protrude outwardly from the outer surface of the body 181 in spaced-apart relationship around the circumference of body 181.

As can be seen, the proximal end of cylindrical body 181 defines an opening and a central cylindrical bore 186 which defines a longitudinal communicating keyway 187. Bore 186 and keyway 187 allow an insertion tool similar in structure to the tool shown in FIG. 8, to be inserted into cylindrical body 181. A key pocket (not shown) similar in structure to the pocket 83 in plate 80, allows the pin of the insertion tool to bear on the left or right interior surface of the key pocket and screw stabilization device 180 in or out of the tunnel in the disc or adjust its position within the tunnel in the disc in a manner similar to that described in connection with the earlier device embodiments.

As seen in FIG. 19, yet another spinal stabilization device 190 embodiment comprises a solid, generally oval-shaped body 191, with a rounded distal end face or nose 192 to distract the vertebra and a flat proximal end face 193 with rounded peripheral edges 194. As described above in connection with FIG. 18, spinal stabilization device 190 may be made of a metal such as titanium, or a material such as high density polyethylene, PEEK or a carbon fiber composite. The device 190 is similar in structure to the device 180 and defines a bore 196, keyway 197 and key pocket (not shown) which have the same structure and function as the similar elements described above in connection with device 180.

In this embodiment, oval body 191 of spinal stabilization device 190 has a number of spikes or studs 195 formed and extending outwardly from the outer surface of the body 191. As shown, studs 195 are arranged helically so as to function as interrupted threads and are pointed at an angle of 90° relative to the exterior surface of oval body 191. Alternatively, studs 195 can be inclined at any desired angle, such as up to about 45° from the perpendicular, preferably only up to about 25° therefrom.

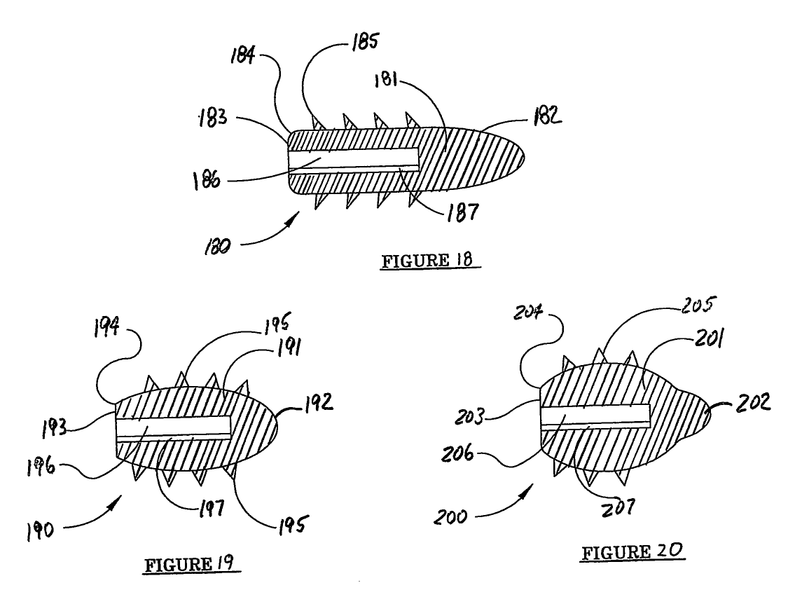

As illustrated in FIG. 20, spinal stabilization device 200 comprises a solid, generally round or bulbous body 201, with a bullet shaped distal end face or nose 202 and a flat proximal radial end face 203 with rounded peripheral edges 204. Spinal stabilization device 200 can be made of the same metal or materials described above with respect to the FIG. 18 embodiment. Body 201 defines a bore 206, keyway 207 and key pocket (not shown) similar in structure to that described above with respect to device 180. In this embodiment, studs or spikes 205 or threads are evenly disposed on the exterior of body 201 and not arranged in a pattern to function as interrupted threads.

FIG. 21 illustrates another spinal stabilization device embodiment 210 whose body 211 is generally shaped to match the shape of the lordotic space between the vertebra above and below the degenerated disc to be treated. Body 211 terminates into a rounded distal end face or nose 212 to distract the vertebra and a flat proximal radial end face 213 with rounded peripheral edges 214. Rounded distal end 212 is substantially smaller in diameter than flat proximal end which defines radial end face 213. Again, spinal stabilization device 210 can be made of the metal or materials described with respect to the FIG. 18 embodiment above. Body 211 defines a bore 216, keyway 217, a key pocket (not shown), and outer studs or spikes 215 similar in structure and function to the corresponding elements described in connection with the device 180. It is understood that, alternatively, bullet-shaped, rounded end 212 of body 211 may be located on the wider end of the body 211, and the narrower end of body 211 may include central keyway 216, key channel 217 and key pocket (not shown) to enable stabilization device 210 to fit within the lordotic space between the patient's vertebra, if the angle of incline of the same so demands.

As shown in FIG. 22, body 211 of spinal stabilization device 210 has rounded side surfaces 218 and flat top and bottom surfaces 219. Helical threads 225 extend outwardly about the rounded side surfaces 218 only and function as interrupted threads. Studs or spikes 215 a are evenly disposed on the flat top and bottom surface 219 of body 211 and are not positioned to act as interrupted threads. Flat top and bottom surfaces 219 provide for greater stability of the stabilization device 210 between the vertebra (not shown). Rounded side surfaces 218 and threads 225 allow the stabilization device 210 to be screwed into place in the tunnel in the disc (not shown).

The top and bottom surfaces of spinal stabilization devices 180, 190 and 200 shown in FIGS. 18-20 can likewise be flattened to enhance the stability of the respective devices between the vertebra. Optionally, the studs or spikes may be evenly disposed on the flattened surfaces and helical threads can be disposed on their rounded side surfaces, as described above.

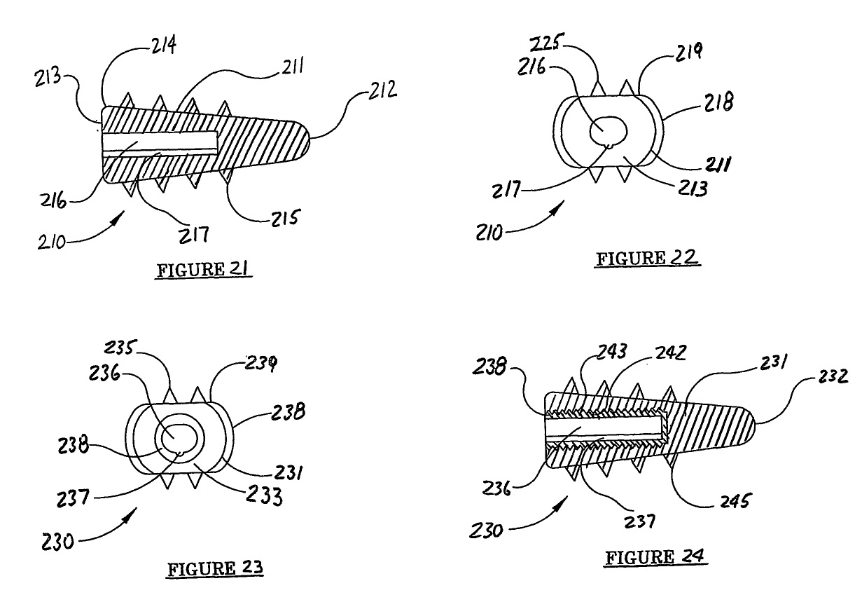

As shown in FIG. 23, the spinal stabilization device 230 instead of being made entirely of a material such as high density polyethylene, PEEK or a carbon fiber composite, may be composed of a body made of several parts. An exterior portion 231 of the body of stabilization device 230 can be made of a material such as a high density polyethylene, PEEK or a carbon fiber composite while an interior portion 238 of the body of spinal stabilization device 230 which is surrounded by the exterior portion 231 can be made of a metal, such as titanium, a titanium alloy or stainless steel. Interior portion 238 defines a bore 236, a keyway 237, and a key pocket (not shown) similar in structure and function to the same corresponding elements described in the FIG. 18 embodiment above. All of the other features and elements of the device 230 are similar to those described above with respect to the device embodiment 210. Likewise, the spinal stabilization devices 180, 190, 200 and 210 can have a composite body made of physiologically compatible materials.

Metallic interior portion 238 of spinal stabilization device 230 can be seen by fluoroscopy (x-ray), enabling the position of stabilization device 230 in the disc to be more accurately ascertained. Materials such as high density polyethylene, PEEK or typical carbon fiber composites, cannot be seen by fluoroscopy. Also, metallic interior portion 238 is less subject to deformation or damage than plastic or carbon fiber composites when being screwed or tapped into place in the tunnel in the disc.

While metallic interior portion 238 can be force-fitted into plastic or carbon fiber composite exterior portion 231 of spinal stabilization device 230, preferably interior portion 238 and exterior portion 231 of stabilization device 230 are threadingly engaged.

As shown in FIG. 24, threads 242 formed on the inner surface of plastic or carbon fiber composite exterior portion 231 of spinal stabilization device 230 engage threads 243 formed on the outer surface of metallic interior portion 238, which defines the bore 236, keyway 237, and the key pocket (not shown).

To prevent excessive angular movement of the vertebra above and below the disc into which the spinal stabilization device has been inserted, it is desirable to limit the angular movement of the vertebra to less than about 40°, preferably to less than about 30°, to prevent either of the vertebra pressing upon or pinching the nerves that lic along the outside perimeter of the disc.

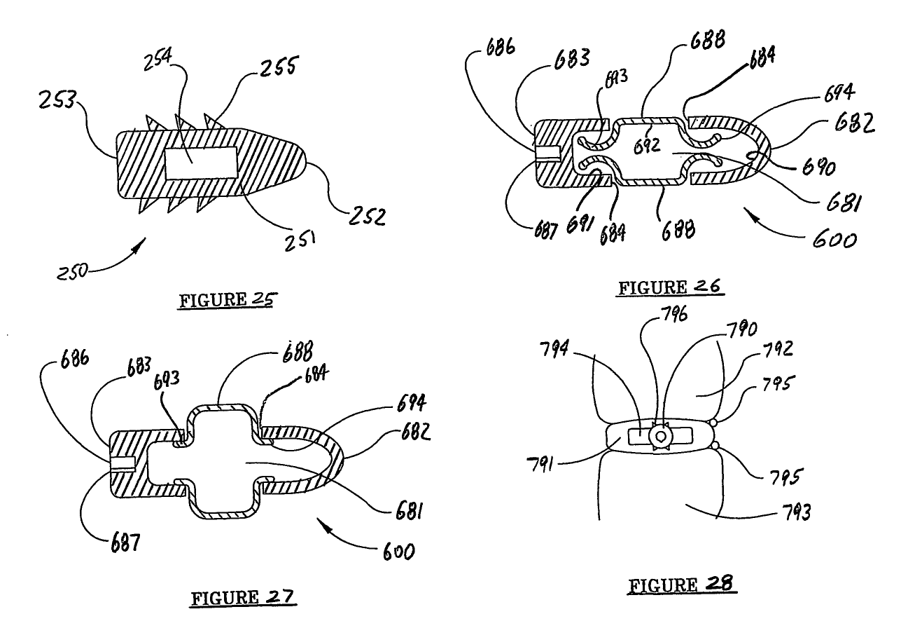

As shown in FIG. 25, cylindrical spinal stabilization device 250 has horizontal slats 254 extending through both sides of a partially hollow body 251 which includes a bullet-shaped distal end 252 and a flat radial proximal end face 253. Slats 254 can likewise extend through both sides of the bodies of the respective spinal stabilization device embodiments described above. The other features and elements of the device embodiment 250 are otherwise similar to the features and elements of the device embodiment 180 shown in FIG. 18.

As shown in FIG. 26, yet another device embodiment 600 is composed of a body 681 including separate, proximal, generally bullet-shaped, nose piece 682, distal end piece or plate 683, and elongate bands 688 extending within and between the pieces 682 and 683. The nose piece 682 defines an interior, hollow cavity 690 while the distal end piece 683 defines and interior, hollow cavity 691. Each of the elongate bands 688 includes a central body portion 692 protruding through the space or slots 684 defined between the pieces 682 and 683 and hooks 693 and 694 formed at the respective ends thereof which extend into the cavities defined in the pieces 682 and 683 respectively. Each of the bands 688 preferably has two-layers of a shaped memory metal, such as a nickel-titanium alloy. The first layer of band 688 has been heat treated to assume a first shape at a temperature less than, for example, 80° F. (about 23.5° C.), and the second layer of band 688 has been heat treated to assume a second shape at a temperature, for example, greater than 80° F. (about 23.5° C.), as known in the art. Hooks 693 and 694 at the end of bands 688 prevent the full egress of the bands 688 out of the pieces 682 and 683. In this embodiment, bore 686, keyway 687, and key pocket (not shown) extend only a short distance inwardly into the end of the piece 683 defining the cavity 691 of stabilization device 600. Alternatively, each band 688 can be made of a single sheet of a shaped memory metal.

FIG. 27 illustrates the second shape of bands 688 of the stabilization device 600 of FIG. 26 a few minutes after spinal stabilization device 600 has been inserted into a disc and it has risen to a temperature, for example, greater than 80° F. (about 23.5° C.). Bands 688 have changed to their second shape and, the body portions 692 thereof extend and bulge outwardly out through the space defined between the pieces 682 and 683 of slats 254, but the hooks 693 and 694 at the ends of bands 688 prevent their full egress out of the pieces 682 and 683. Bands 688 limit the degree of movement of the vertebra (not shown) above and below the degenerated disc (not shown) into which spinal stabilization device 600 has been inserted.

FIG. 28 illustrates the placement of another spinal stabilization device embodiment 790 within disc 791 between vertebra 792 and 793. Prior to insertion into the tunnel (not shown) in disc 791, stabilization device 790 has been cooled to a temperature substantially below about 80° F. (about 23.5° C.), preferably to a temperature of about 350 to 50° F. (about 2° to 10° C.). When stabilization device is warmed by the body to a temperature above, for example, 80° F. (about 23.5° C.), bands 794 assume their second shape and extend out of the stabilization device 790, limiting the angle at which vertebra 792 and 793 can move toward each other, avoiding excessive pressure on or the pinching of traversing or exiting nerves 795. Studs or spikes 796 anchor and prevent movement of spinal stabilization device 790 within disc 791 between vertebra 792 and 793.

Lasers which may be used to vaporize bone to open the foraminal space and vaporize intervening tissue to enable the disc, vertebra and nerves to be seen include, but are not limited to, Holmium:YAG, CTH:YAG or Holmium: YSGG lasers, Excimer (excited dimer lasers), pulsed or Q-switched KTP or Nd: YAG lasers and others. Preferably, a Holmium: YAG laser, such as the 80 watt Omnipulse™ MAX Holmium laser manufactured by Trimedyne, Inc. (Irvine, Calif.), which can emit either an evenly time-spaced train of pulses (Single Pulse™ mode), or two pulses close together, separated by doubled time-spaces (Double Pulse™ mode). Said Double Pulse™ mode produces the ablative effect of two pulses, and the doubled time period between pulses allows a longer time for the tissue to cool between pulses. Double Pulse™ mode is preferred for vaporization of bone, as it has sufficient power to rapidly vaporize bone yet avoids thermal damage to the remaining bone. Single Pulse™ mode is preferred for rapid vaporization of soft tissues, such as annulus and nucleus pulposa.

About 40 to 80 watts of energy in Double Pulse™ mode may be used to vaporize bone, about 20 to 40 watts of energy in Single Pulse™ mode may be used to vaporize soft tissues and about 10 to 20 watts of energy in Single Pulse™ mode may be used for coagulation of bleeding.

While a preferred method of placement of the spinal stabilization device entails the earlier creation of a bore or tunnel into the disc, all or most of the nucleus pulposa, if diseased, may be removed. Alternatively, instead of using laser energy or a mechanical burr, auger or drill to make an opening in the annulus, a sharp-ended tool may be used to puncture the annulus, after which, without making a bore or tunnel into the disc, the spinal stabilization device may be introduced directly through the puncture in the annulus into place in the disc. Alternatively, if the spinal stabilization device has a sharply pointed distal end, it may be inserted directly into an intact disc, using its sharp end to puncture the annulus, without earlier creating a puncture, bore or tunnel into the disc.

To date, fifteen human patients with degenerated lumbar discs have been treated on a minimally invasive, outpatient basis using the method described herein. A spinal stabilization device, such as shown in FIGS. 9 and 10 above, with an outside diameter, including its threads, of 8 mm to 10 mm, depending on the patient's anatomy, and a length of about 24 to 27 mm, was utilized for the treatment.

Following the injection of a local anesthetic, posterolateral insertion of a guide wire into the disc, dilation of a passageway about 5 mm in diameter and insertion of an endoscope up to the facet of the spine, the Omnipulse™ MAX 80 watt Holmium laser manufactured by Trimedyne, Inc. (Irvine, Calif.) was used through the Vapor MAX™ side firing laser needle manufactured by Trimedyne, Inc. to vaporize bone of the facet to create an opening into the foraminal space, enabling the endoscope to be inserted and the disc, vertebra and nerves to be seen. Using mechanical tools, a tunnel was made into the disc, about 26 to 28 mm in length, with an inside diameter of about 8 to 10 mm, and additional disc material was optionally removed, an anesthetic was injected into the end plates of the vertebra, which were subsequently scraped to promote oxygen and nutrient delivery to the disc.

A delivery cannula was inserted up to the disc, and one spinal stabilization device was screwed into place in the tunnel in the disc, as described in greater detail above. The delivery cannula was removed, the endoscope was reinserted and the laser needle was used to shrink the annulus of the disc and to vaporize debris and coagulate bleeding. The endoscope was removed, and one stitch and a Band Aid® (Johnson & Johnson, New Brunswick, N.J.) were applied. The lower back and leg pain usually disappeared or was significantly reduced near the end of the procedure or in the recovery room. The patients where able to walk out of the facility and resume light daily activities in a few days (light manual labor in 2 to 10 weeks). Post-operative pain from the puncture in the back was minimal.

X-rays were made prior to, immediately after and at 1, 3, 6 and 12 months following the procedures. None of the spinal stabilization devices had moved, there was no subluxation (relative movement) of the vertebra, and there was no evidence of erosion of the vertebra above and below the disc in which the spinal stabilization device had been implanted. The procedure was deemed successful (pain questionnaires yielding excellent or good results) in 80% (12) of the patients, fair in 13% (2) and no change in 7% (1). These results are significantly better than the 40-77% success rates (based on results of similar pain questionnaires) reported in the literature for conventional fusion surgery to treat degenerated lumbar discs, which entails the adverse effects and high cost described above.

Degenerated lumbar discs can likewise be treated using a posterolateral approach and an expandable spinal stabilization device, as described above. Degenerated lower thoracic discs can be treated from a posterolateral approach, using the method described above and a non-expandable or expandable spinal stabilization device, or from an anterolateral approach. Degenerated upper thoracic and cervical discs can also be treated in a minimally invasive, outpatient procedure, using an anterolateral approach and a non-expandable or expandable spinal stabilization device, as described herein.

Numerous variations and modifications of the embodiments described above can be effected without departing from the spirit and scope of the novel features of the invention. It is to be understood that no limitation with respect to the specific devices or methods illustrated herein is intended or should be inferred. It is, of course, intended to cover by the appended claims all such modifications as fall within the scope of the claims.

1. A spinal stabilization device comprising:
a cage having a distal end adapted for insertion into a spinal disc passageway formed in a spinal disc and an open proximal end adapted to receive the shaft of an insertion device; and
anchors on the exterior of the cage for stabilizing the cage in the spinal disc passageway.

2. The device in accordance with claim 1 wherein the cage is made of a helical metal coil having exterior edges beveled into a sharp point and comprising the anchors, the coil further including internal threads formed adjacent the proximal and distal ends thereof and adapted to receive an end plate and a nose piece respectively, the nose piece being closed for extension into the passageway formed in the spinal disc and the end plate being open to receive the shaft of the insertion device.

3. The device in accordance with claim 1 wherein the cage is made of a hollow metal cylinder having helical threads extending around an outer surface thereof and comprising the anchors, the cylinder further defining a plurality of columns of openings formed therein and extending around the circumference thereof between each of the helical threads, the cylinder further including internal threads formed in proximal and distal ends thereof for threadingly securing an end plate and a nose piece thereto respectively, the nose piece being closed for extension into the passageway formed in the spinal disc and the end plate being open to receive the shaft of the insertion device.

4. The device in accordance with claim 1 wherein the cage is made of a hollow metal cone having helical threads extending around an outer surface thereof and comprising the anchors, the cylinder further defining a plurality of columns of openings formed therein and extending around the circumference thereof between each of the helical threads, the cylinder further including internal threads formed in proximal and distal ends thereof for threadingly securing thereto an end plate and a nose piece respectively, the nose piece being closed for extension into the passageway formed in the spinal disc and the end plate being open to receive the shaft of the insertion device.

5. The device in accordance with claim 1 wherein the cage is a hollow sphere having a plurality of longitudinally extending ridges thereon comprising the anchors and a plurality of rows of generally oval shaped openings defined on the exterior surface of the sphere, the sphere further including internal threads formed in the proximal and distal ends thereof for threadingly receiving an end plate and a nose piece respectively, the nose piece being closed for extension into the passageway formed in the spinal disc and the end plate being open to receive the shaft of the insertion device.

6. The device in accordance with claim 1 wherein the cage is defined by a plurality of longitudinally oriented elongate slats extending between a nose piece and an end plate respectively, the end plate defining a bore and the nose piece defining an interior recess, the cage further including a screw extending through the bore in the end plate and the recess in the nose piece and being operably associated therewith such that when the screw is rotated in response to the rotation of the insertion or screwing device the end plate is moved in the direction of the nose piece and the slats flex outwardly, each of the slats having a plurality of spikes formed thereon and comprising the anchors.

7. The device in accordance with claim 1 wherein the cage is defined by a plurality of longitudinal, elongate slats extending between a nose piece and an end plate, the nose piece including a closed, rounded distal end and the end plate including an open proximal end adapted to receive the shaft of the insertion or screwing device, the slats being made of a shape memory alloy and adapted to flex outwardly in response to a change in temperature.

8. The device in accordance with claim 1 further comprising inner slats extending between the end plate and a proximal end of the nose piece and made of a shape memory alloy adapted to contract in response to a change in temperature.

9. The device in accordance with claim 3 wherein the hollow metal cylinder is made from a rolled sheet of flat metal.

10. The device in accordance with claim 1 wherein the cage is defined by a body having a bore defining an opening in the open proximal end thereof and a key hole adapted to receive the shaft of the insertion or screwing device.

11. The device in accordance with claim 10 wherein the distal end of the cage is generally bullet-shaped.

12. The device in accordance with claim 10 wherein the distal end of the cage is generally rounded.

13. The device in accordance with claim 10 wherein the body of the cage is generally bulbous and the distal end thereof is generally bullet-shaped.

14. The device in accordance with claim 10 wherein the cage is generally cone-shaped.

15. The device in accordance with claim 10 wherein a plurality of spikes extend outwardly from an outer surface of the body and define the anchors.

16. The device in accordance with claim 15 wherein the spikes extend from upper and lower faces of the outer surface of the body and threads extend outwardly from side faces of the outer surface of the body.

17. The device in accordance with claim 10 wherein the body is composed of an inner core made of a first material and an outer core made of a second material.

18. The device in accordance with claim 17 wherein the inner core is threaded within the outer core.

19. The device in accordance with claim 1 wherein the cage comprises a separate nose piece and end plate defining respective interior cavities and bands composed of shape memory alloy extending between the nose piece and the end plate.

20. The device in accordance with claim 1 wherein the bands include hooked ends extending into the cavities defined in the nose piece and the end plate respectively.

21. The device in accordance with claim 1 wherein the cage is packed with bone growth stimulating agents.

22. The device in accordance with claim 1 wherein the cage is packed with a patient's bone marrow.

23. The device in accordance with claim 22 wherein the bone marrow is mixed with a thixotropic material.

24. The device in accordance with claim 22 wherein the bone marrow is mixed with bovine or other collagen.

25. A spinal stabilization device which comprises:
an expandable cage having a closed, rounded distal end and an open proximal end;
said cage being provided with plural anchors on the external surface thereof for engagement with contiguous bone tissue when said cage is expanded.

26. The device in accordance with claim 25 wherein the cage is made of a memory metal and expands to a predetermined shape at body temperature.

27. The device in accordance with claim 25 wherein the cage is expandable mechanically.

28. The device in accordance with claim 25 wherein an end plate is attached to the proximal end of the cage.

29. A method of treating a degenerated disc comprising the steps of creating an opening in the foraminal space using either a posterolateral or anterolateral approach and implanting a spinal stabilization device through the opening in the foraminal space and into the degenerated disc.

30. The method of claim 29 wherein the step of implanting the spinal stabilization device into the degenerated disc includes the steps of using laser energy to create an opening in the annulus, using a sharp-ended tool to create a puncture in the annulus and threading the spinal stabilization device through the puncture and into place in the disc.

31. The method of claim 29 wherein the step of implanting the spinal stabilization device into the degenerated disc includes the steps of forming a tunnel in the disc and then inserting the spinal stabilization device into the tunnel.

32. The method of claim 29 wherein the spinal stabilization device has a sharp end and the step of implanting the spinal stabilization device includes the step of using the sharp end to puncture the annulus and then inserting the spinal stabilization device into the disc through the puncture.

33. The method of claim 29 further including the steps of inserting a laser needle posterolaterally to vaporize bone and inserting an endoscope to enable viewing of the disc, vertebra and nerves prior to the step of creating an opening in the foraminal space.

34. The method of claim 29 further including the step of using a laser needle to shrink the annulus of the disc and to vaporize debris following the step of implanting the spinal stabilization device.

35. A method for stabilizing the spine of a human patient which comprises the steps of:
forming a passageway in a spinal disc;
inserting an expandable spinal stabilization device into the formed passageway; and
expanding the inserted spinal stabilization device while in the formed passageway sufficient to stabilize the spine.
